(19) United States
(12) Patent Application Publication
(10) Pub. No.: US 2016/0096810 A1
(43) Pub. Date: Apr. 7, 2016

(54) ENANTIOSELECTIVE SYNTHESIS OF DIALKYLATED N,O-HETEROCYCLES BY PALLADIUM-CATALYZED ALLYLIC ALKYLATION

(71) Applicants: Brian M. Stoltz, San Marino, CA (US); Yoshitaka Numajiri, Kanagawa (JP); Douglas C. Behenna, Laguna Niguel, CA (US)

(72) Inventors: Brian M. Stoltz, San Marino, CA (US); Yoshitaka Numajiri, Kanagawa (JP); Douglas C. Behenna, Laguna Niguel, CA (US)

(21) Appl. No.: 14/877,496

(22) Filed: Oct. 7, 2015

Related U.S. Application Data
(60) Provisional application No. 62/060,922, filed on Oct. 7, 2014.

(51) Int. Cl. C07D 265/32; C07D 265/36; C07D 265/10; C07D 261/04; C07D 265/02; C07D 267/04; C07D 279/12; C07D 263/18

(52) U.S. Cl. CPC C07D 265/32; C07D 279/12; C07D 265/36; C07D 263/18; C07D 261/04; C07D 265/02; C07D 267/04; C07D 265/10

(57) ABSTRACT

This invention provides enantioenriched N,O-heterocyclic compounds with quaternary stereogenic centers and novel methods of preparing the compounds. Methods include the method for the preparation of a compound of formula (I):

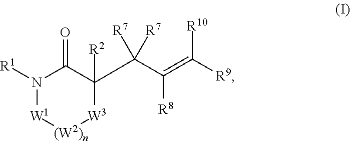

comprising treating a compound of formula (II):

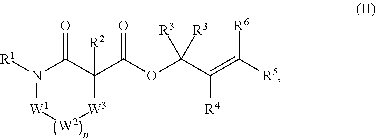

with a transition metal catalyst under alkylation conditions.

### RELATED APPLICATIONS

This application claims the benefit of U.S. Provisional Application 62/060,922, filed Oct. 7, 2014, the content of which is incorporated herein by reference.

### GOVERNMENT SUPPORT

This invention was made with Government support under Grant Number R01GM080269, awarded by the National Institutes of Health. The Government has certain rights in the invention.

### BACKGROUND OF THE INVENTION

N,O-heterocycles such as morpholine, oxazolidine, and isoxazolidine are important pharmacophores in medicinal chemistry. Notable morpholine-containing pharmaceuticals include edivoxetine, an antidepressant and a treatment for ADHD; linezolid, a synthetic antibiotic; gefitinib, an EGFR inhibitor used to treat certain breast, lung and other cancers. 5-Membered isoxazolidinone is the core structure of cycloserine, an antibiotic for the treatment of tuberculosis. Quinocarcin, possessing an oxazolidine ring in the 3,8-diazabicyclo[3.2.1]octane framework, has shown remarkable antiproliferative activity against lymphocytic leukemia. An antibiotic, FR-66979, isolated from Streptomyces sandaensis No. 6897, contains a 1,2-oxazinane moiety.

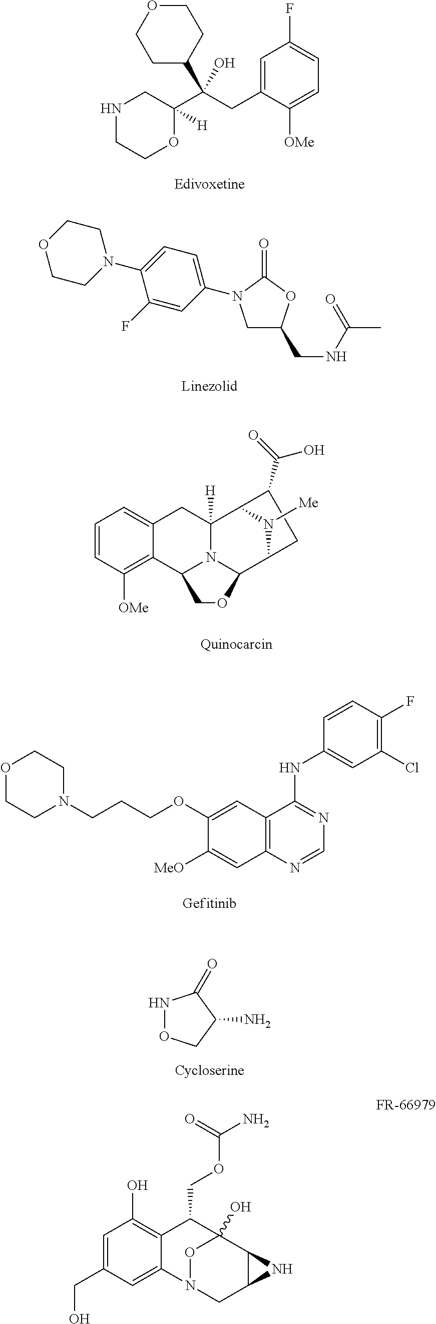 [SMILES: COc1ccc(F)cc1C[C@](O)(C1CCOCC1)[C@@H]1CNCCO1 ; CC(=O)NC[C@H]1CN(c2ccc(N3CCOCC3)c(F)c2)C(=O)O1 ; COc1cccc2c1[C@@H]1CO[C@@H]3[C@@H]4C[C@@H](C(=O)O)[C@H]([C@H](C2)N31)N4C ; COc1cc2ncnc(Nc3ccc(F)c(Cl)c3)c2cc1OCCCN1CCOCC1 ; N[C@@H]1CONC1=O ; NC(=O)OC[C@H]1c2c(O)cc(CO)cc2N2C[C@@H]3N[C@@H]3C1(O)O2]

In addition to a wide variety of biological activity, N,O-heterocycles are commonly used as synthetic intermediates, which provide various hydroxy acid moieties after acetal removal or N—O bond cleavage. Oxazolidin-4-ones have been reported as good platforms to α-hydroxyacids. For example, Ye et al. reported that the formal [3+2] cycloaddition between ketenes and oxaziridines could be applied in an enantioselective fashion to synthesize oxazolidin-4-one derivatives, which could be converted into the corresponding α-hydroxy acids. 1,2-Oxazinan-3-ones have proved to be excellent precursors to γ-hydroxy acid and γ-butyrolactone derivatives.

There is a need for methods that would allow access to N,O-heterocycle scaffolds bearing stereogenic centers, particularly enantioselective methods to provide enantioenriched products.

### SUMMARY OF THE INVENTION

In one aspect, the present invention provides methods for preparing a compound of formula (I):

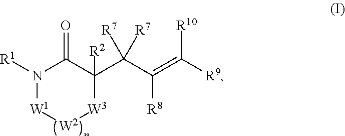

comprising treating a compound of formula (II):

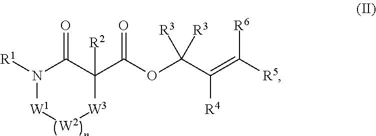

with a transition metal catalyst under alkylation conditions, wherein, as valence and stability permit,
R¹ represents hydrogen or optionally substituted alkyl, cycloalkyl, (cycloalkyl)alkyl, aryl, aralkyl, heteroaryl, heteroaralkyl, alkenyl, alkynyl, —C(O)alkyl, —C(O)aryl, —C(O)aralkyl, —C(O)heteroaryl, —C(O)heteroaralkyl, —C(O)O(alkyl), —C(O)O(aryl), C(O)O(aralkyl), —C(O)O(heteroaryl), or —C(O)O(heteroaralkyl); or
R² represents hydrogen or substituted or unsubstituted alkyl, aralkyl, aryl, heteroaralkyl, heteroaryl, alkenyl, alkynyl, or halo;
R³, R⁴, R⁵, R⁶, R⁷, R⁸, R⁹, and R¹⁰ are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
W¹, W², and W³ are independently selected for each occurrence from CR¹¹R¹², O, and S;
R¹¹ and R¹² are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
wherein any occurrence of R¹¹ may optionally combine with any second occurrence of R¹¹ or an occurrence of R¹², along with the carbons to which they are attached, to form an optionally substituted 3-8 membered ring; and
n is an integer from 0-3;
wherein the compound of formula (I) is not

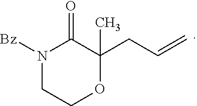

In another aspect, the present invention provides compounds of formula (I):

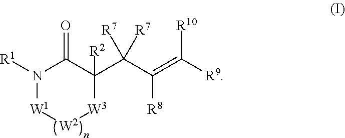

In another aspect, the present invention provides compounds of formula (II):

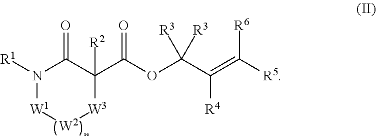

### DETAILED DESCRIPTION OF THE INVENTION

### I. Definitions

The definitions for the terms described below are applicable to the use of the term by itself or in combination with another term.

The term “acyl” is art-recognized and refers to a group represented by the general formula hydrocarbyl-C(O)—, preferably alkyl-C(O)—.

The term “acylamino” is art-recognized and refers to an amino group substituted with an acyl group and may be represented, for example, by the formula hydrocarbyl-C(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond that is straight chained or branched and has from 1 to about 20 carbon atoms, preferably from 1 to about 10 unless otherwise defined. The term “alkenyl” is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl such as an alkylC(O)), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a silyl ether, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthiols, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkyl-S—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

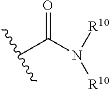

wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

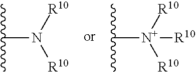

wherein each R¹⁰ independently represents a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group. An aralkyl group is connected to the rest of the molecule through the alkyl component of the aralkyl group.

The term “aryl” as used herein include substituted or unsubstituted single-ring aromatic groups in which each atom of the ring is carbon. Preferably the ring is a 5- to 10-membered ring, more preferably a 6- to 10-membered ring or a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like. Exemplary substitution on an aryl group can include, for example, a halogen, a haloalkyl such as trifluoromethyl, a hydroxyl, a carbonyl (such as a carboxyl, an alkoxycarbonyl, a formyl, or an acyl such as an alkylC(O)), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a silyl ether, a sulfhydryl, an alkylthio, a sulfate, a sulfonate, a sulfamoyl, a sulfonamido, a sulfonyl, a heterocyclyl, an aralkyl, or an aromatic or heteroaromatic moiety

The term “carbamate” is art-recognized and refers to a group

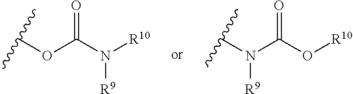

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxyl”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a heteroaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include 5- to 10-membered cyclic or polycyclic ring systems, including, for example, pyrrole, furan, thiophene, imidazole, oxazole, thiazole, pyrazole, pyridine, pyrazine, pyridazine, and pyrimidine, and the like. Exemplary optional substituents on heteroaryl groups include those substituents put forth as exemplary substituents on aryl groups, above.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto. A “silyl ether” refers to a silyl group linked through an oxygen to a hydrocarbyl group. Exemplary silyl ethers include —OSi(CH₃)₃ (—OTMS), —OSi(CH₃)₂t-Bu (—OTBS), —OSi(Ph)₂t-Bu (—OTBDPS), and —OSi(iPr)₃ (—OTIPS).

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a haloalkyl, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

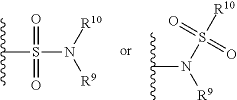

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

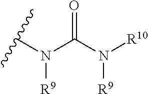

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

### II. Description of the Invention

This invention is based on the discovery that catalytic decarboxylative allylic alkylation reactions in N,O-heterocyclic substrates generate N,O-heterocyclic products with α-tertiary and α-quaternary stereocenters, proceeding in high yield and enantioselectivity. The decarboxylative allylic alkylation reaction is catalyzed by a transition metal catalyst and a chiral ligand, and the products can be quickly and efficiently elaborated into complex products exhibiting biological activity.

According to embodiments of the present invention, a wide range of structurally-diverse, functionalized N,O-heterocyclic compounds are prepared by a stereoselective method of palladium-catalyzed enantioselective enolate allylic alkylation. This chemistry is important to the synthesis of bioactive alkaloids, and the transformation is useful for the construction of novel building blocks for medicinal and polymer chemistry. Indeed, in some embodiments of the present invention, these novel building blocks include N,O-heterocyclic compounds useful as precursors to (or reactants leading to the preparation of) numerous biologically active and important natural and pharmaceutical products. While embodiments of the present invention are directed to the novel building blocks achieved from the transition-metal catalyzed allylic alkylation reaction, other embodiments of the present invention are directed to novel N,O-heterocyclic substrates used in the transition-metal catalyzed allylic alkylation reaction to form the building blocks.

Indeed, in some embodiments of the present invention, a method of making a building block compound comprises reacting a substrate compound with a ligand in the presence of a palladium-based catalyst and a solvent. The palladium-based catalysts, ligands and solvents useful in this reaction are described in more detail below in Section III.

### III. Compounds and Methods of the Invention

In certain embodiments, the invention relates to a compound of formula (I),

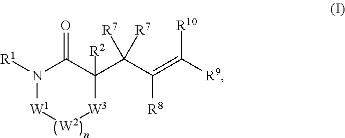

R¹ represents hydrogen or optionally substituted alkyl, cycloalkyl, (cycloalkyl)alkyl, aryl, aralkyl, heteroaryl, heteroaralkyl, alkenyl, alkynyl, —C(O)alkyl, —C(O)aryl, —C(O)aralkyl, —C(O)heteroaryl, —C(O)heteroaralkyl, —C(O)O(alkyl), —C(O)O(aryl), C(O)O(aralkyl), —C(O)O(heteroaryl), or —C(O)O(heteroaralkyl); or
R² represents hydrogen or substituted or unsubstituted alkyl, aralkyl, aryl, heteroaralkyl, heteroaryl, alkenyl, alkynyl, or halo;
R⁷, R⁸, R⁹, and R¹⁰ are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
W¹, W², and W³ are independently selected for each occurrence from CR¹¹R¹², O, and S;
R¹¹ and R¹² are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
wherein any occurrence of R¹¹ may optionally combine with any second occurrence of R¹¹ or an occurrence of R¹², along with the carbons to which they are attached, to form an optionally substituted 3-8 membered ring; and
n is an integer from 0-3;
wherein the compound of formula (I) is not

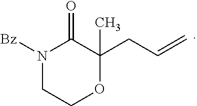

In certain embodiments, W¹ is O. In certain such embodiments, W² and W³ each represent CR¹¹R¹², for example W² and W³ can each represent CH₂. In certain such embodiments, n is selected from 0, 1 and 2.

In certain embodiments, W² is O and n is 1. In certain such embodiments, W¹ and W³ each represent CR¹¹R¹²,e.g., wherein R¹¹ and R¹² are both hydrogen, or wherein at least one occurrence of R¹¹ or R¹² is not hydrogen.

In certain embodiments, W³ is O. In certain such embodiments, W¹ and W² each represent CR¹¹R¹², e.g., wherein R¹¹ and R¹² are both hydrogen, or wherein at least one occurrence of R¹¹ or R¹² is not hydrogen. In certain such embodiments, n is selected from 0, 1, and 2.

In certain embodiments, R⁷, R⁸, R⁹, and R¹⁰ are each independently selected for each occurrence from hydrogen, halogen, cyano, alkyl, alkoxy, alkylthio, amide, amine, aryloxy, and arylalkoxy.

In certain embodiments, R⁷, R⁸, R⁹, and R¹⁰ are each hydrogen.

In certain embodiments, R² is selected from optionally substituted alkyl, aryl, aralkyl, haloalkyl, alkoxyalkyl, and hydroxyalkyl.

In certain embodiments, R² is alkyl, optionally substituted with halo, hydroxy, alkoxy, aryloxy, arylalkoxy, cyano, nitro, azido, —CO₂H, —C(O)O(alkyl), or amino.

In certain embodiments, R¹ represents optionally substituted alkyl, cycloalkyl, (cycloalkyl)alkyl, aryl, aralkyl, heteroaryl, heteroaralkyl, alkenyl, alkynyl, —C(O)alkyl, —C(O)aryl, —C(O)aralkyl, —C(O)heteroaryl, —C(O)heteroaralkyl, —C(O)O(alkyl), —C(O)O(aryl), C(O)O(aralkyl), —C(O)O(heteroaryl), or —C(O)O(heteroaralkyl).

In certain embodiments, R¹ represents optionally substituted —C(O)alkyl, —C(O)aryl, —C(O)aralkyl, —C(O)heteroaryl, —C(O)heteroaralkyl, —C(O)O(alkyl), —C(O)O(aryl), C(O)O(aralkyl), —C(O)O(heteroaryl), or —C(O)O(heteroaralkyl).

In certain embodiments, R¹¹ and R¹² are each selected, independently for each occurrence, from hydrogen, halogen, cyano, alkyl, alkoxy, alkylthio, amide, amine, aryloxy, and arylalkoxy.

In certain embodiments, R¹¹ and R¹² are each selected, independently for each occurrence, from hydrogen, alkyl, alkoxy, and alkylthio.

In certain embodiments, the compound of formula (I) is enantioenriched.

In certain embodiments, the present invention also relates to a compound represented by formula (II),

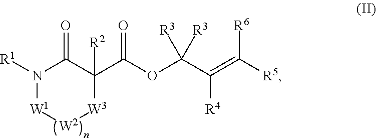

or a tautomer and/or salt thereof, wherein:
R¹ represents hydrogen or optionally substituted alkyl, cycloalkyl, (cycloalkyl)alkyl, aryl, aralkyl, heteroaryl, heteroaralkyl, alkenyl, alkynyl, —C(O)alkyl, —C(O)aryl, —C(O)aralkyl, —C(O)heteroaryl, —C(O)heteroaralkyl, —C(O)O(alkyl), —C(O)O(aryl), C(O)O(aralkyl), —C(O)O(heteroaryl), or —C(O)O(heteroaralkyl); or
R² represents hydrogen or substituted or unsubstituted alkyl, aralkyl, aryl, heteroaralkyl, heteroaryl, alkenyl, alkynyl, or halo;
R³, R⁴, R⁵, and R⁶ are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
W¹, W², and W³ are independently selected for each occurrence from CR¹¹R¹², O, and S;
R¹¹ and R¹² are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
wherein any occurrence of R¹¹ may optionally combine with any second occurrence of R¹¹ or an occurrence of R¹², along with the carbons to which they are attached, to form an optionally substituted 3-8 membered ring; and
n is an integer from 0-3;
wherein the compound is not

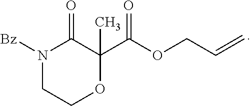

In other aspects, the present invention provides a method for preparing a compound of formula (I):

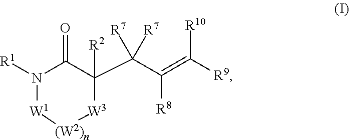

comprising treating a compound of formula (II):

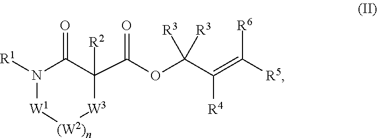

with a transition metal catalyst under alkylation conditions, wherein, as valence and stability permit,
R¹ represents hydrogen or optionally substituted alkyl, cycloalkyl, (cycloalkyl)alkyl, aryl, aralkyl, heteroaryl, heteroaralkyl, alkenyl, alkynyl, —C(O)alkyl, —C(O)aryl, —C(O)aralkyl, —C(O)heteroaryl, —C(O)heteroaralkyl, —C(O)O(alkyl), —C(O)O(aryl), C(O)O(aralkyl), —C(O)O(heteroaryl), or —C(O)O(heteroaralkyl); or
R² represents hydrogen or substituted or unsubstituted alkyl, aralkyl, aryl, heteroaralkyl, heteroaryl, alkenyl, alkynyl, or halo;
R³, R⁴, R⁵, R⁶, R⁷, R⁸, R⁹, and R¹⁰ are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
W¹, W², and W³ are independently selected for each occurrence from CR¹¹R¹², O, and S;
R¹¹ and R¹² are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
wherein any occurrence of R¹¹ may optionally combine with any second occurrence of R¹¹ or an occurrence of R¹², along with the carbons to which they are attached, to form an optionally substituted 3-8 membered ring; and
n is an integer from 0-3;
wherein the compound of formula (I) is not

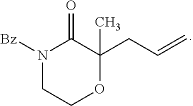

In certain embodiments, W¹ is O. In certain such embodiments, W² and W³ each represent CR¹¹R¹², for example W² and W³ can each represent CH₂. In certain such embodiments, n is selected from 0, 1, and 2.

In certain embodiments, W² is O and n is 1. In certain such embodiments, W¹ and W³ each represent CR¹¹R¹², e.g., wherein R¹¹ and R¹² are both hydrogen, or wherein, at least one occurrence of R¹¹ or R¹² is not hydrogen.

In certain embodiments, W³ is O. In certain such embodiments, W¹ and W² each represent CR¹¹R¹², e.g., wherein R¹¹ and R¹² are both hydrogen, or wherein at least one occurrence of R¹¹ or R¹² is not hydrogen. In certain such embodiments, n is selected from 0, 1, and 2.

In certain embodiments, R³, R⁴, R⁵, R⁶, R⁷, R⁸, R⁹, and R¹⁰ are each independently selected for each occurrence from hydrogen, halogen, cyano, alkyl, alkoxy, alkylthio, amide, amine, aryloxy, and arylalkoxy.

In certain embodiments, R³, R⁴, R⁵, R⁶, R⁷, R⁸, R⁹, and R¹⁰ are each hydrogen.

In certain embodiments, R² is selected from optionally substituted alkyl, aryl, aralkyl, haloalkyl, alkoxyalkyl, and hydroxyalkyl.

In certain embodiments, R² is alkyl, optionally substituted with halo, hydroxy, alkoxy, aryloxy, arylalkoxy, cyano, nitro, azido, —CO₂H, —C(O)O(alkyl), or amino.

In certain embodiments, R¹ represents optionally substituted alkyl, cycloalkyl, (cycloalkyl)alkyl, aryl, aralkyl, heteroaryl, heteroaralkyl, alkenyl, alkynyl, —C(O)alkyl, —C(O)aryl, —C(O)aralkyl, —C(O)heteroaryl, —C(O)heteroaralkyl, —C(O)O(alkyl), —C(O)O(aryl), C(O)O(aralkyl), —C(O)O(heteroaryl), or —C(O)O(heteroaralkyl).

In certain embodiments, R¹ represents optionally substituted —C(O)alkyl, —C(O)aryl, —C(O)aralkyl, —C(O)heteroaryl, —C(O)heteroaralkyl, —C(O)O(alkyl), —C(O)O(aryl), C(O)O(aralkyl), —C(O)O(heteroaryl), or —C(O)O(heteroaralkyl).

In certain embodiments, R¹¹ and R¹² are each selected, independently for each occurrence, from hydrogen, halogen, cyano, alkyl, alkoxy, alkylthio, amide, amine, aryloxy, and arylalkoxy.

In certain embodiments, R¹¹ and R¹² are each selected, independently for each occurrence, from hydrogen, alkyl, alkoxy, and alkylthio.

In certain embodiments, the compound of formula (I) is enantioenriched.

### Transition Metal Catalysts

Preferred transition metal catalysts of the invention are complexes of transition metals wherein the metal is selected from Groups 6, 8, 9 and 10 in the periodic table. In preferred embodiments, the metal of the transition metal catalyst is selected from molybdenum, tungsten, iridium, rhenium, ruthenium, nickel, platinum, and palladium. In more preferred embodiments, the transition metal catalyst comprises a transition metal selected from palladium, nickel, and platinum, most preferably palladium.

In certain embodiments of the invention, the transition metal complex utilized in the reaction includes a transition metal that has a low oxidation state, typically (0) or (I). A low oxidation state enables the metal to undergo oxidative addition to the substrate. It should be appreciated that the air- and moisture-sensitivity of many such complexes of transition metals will necessitate appropriate handling precautions. This may include the following precautions without limitation: minimizing exposure of the reactants to air and water prior to reaction; maintaining an inert atmosphere within the reaction vessel; properly purifying all reagents; and removing water from reaction vessels prior to use.

Exemplary transition metal catalysts include, without limitation, Mo(CO)₆, Mo(MeCN)₃(CO)₃, W(CO)₆, W(MeCN)₃(CO)₃, [Ir(1,5-cyclooctadiene)Cl]₂, [Ir(1,5-cyclooctadiene)Cl]₂, [Ir(1,5-cyclooctadiene)Cl]₂, Rh(PPh₃)₃Cl, [Rh(1,5-cyclooctadiene)Cl]₂, Ru(pentamethylcyclopentadienyl)(MeCN)₃PF₆, Ni(1,5-cyclooctadiene)₂, Ni[P(OEt)₃]₄, Tris(dibenzylideneacetone)dipalladium(0), tris(dibenzylideneacetone)dipalladium(0)-chloroform Adduct, bis(4-methoxybenzylidene)acetone)dipalladium(0), Pd(OC(═O)CH₃)₂, Pd(3,5-dimethyoxy-dibenzylideneacetone)₂, PdCl₂(R²³CN)₂; PdCl₂(PR²⁴R²⁵R²⁶)₂; [Pd(η³-allyl)Cl]₂; and Pd(PR²⁴R²⁵R²⁶)₄, wherein R²³, R²⁴, R²⁵, and R²⁶ are independently selected from hydrocarbyl, substituted hydrocarbyl, heteroatom-containing hydrocarbyl, and substituted heteroatom-containing hydrocarbyl. In particular embodiments, the transition metal catalyst tris(dibenzylideneacetone)dipalladium, Pd₂(dba)₃, or bis(4-methoxybenzylidene)acetone)dipalladium, Pd₂(pmdba)₃, is preferred.

To improve the effectiveness of the catalysts discussed herein, additional reagents may be employed as needed, including, without limitation, salts, solvents, and other small molecules. Preferred additives include AgBF₄, AgOSO₂CF₃, AgOC(═O)CH₃, and bipyridine. These additives are preferably used in an amount that is in the range of about 1 equivalent to about 5 equivalents relative to the amount of the catalyst.

A low oxidation state of a transition metal, i.e., an oxidation state sufficiently low to undergo oxidative addition, can also be obtained in situ, by the reduction of transition metal complexes that have a high oxidation state. Reduction of the transition metal complex can be achieved by adding nucleophilic reagents including, without limitation, NBu₄OH, tetrabutylammonium difluorotriphenylsilicate (TBAT), tetrabutylammonium fluoride (TBAF), 4-dimethylaminopyridine (DMAP), NMe₄OH(H₂O)₅, KOH/1,4,7,10,13,16-hexaoxacyclooctadecane, EtONa, TBAT/trimethyl-(2-methyl-cyclohex-1-enyloxy)-silane, and mixtures thereof. When a nucleophilic reagent is needed for the reduction of the metal complex, the nucleophilic reagent is used in an amount in the range of about 1 mol % to about 20 mol % relative to the reactant, more preferably in the range of about 1 mol % to about 10 mol % relative to the substrate, and most preferably in the range of about 5 mol % to about 8 mol % relative to the substrate.

In certain embodiments, a Pd(II) complex can be reduced in situ to form a Pd(0) catalyst. Exemplary transition metal complexes that may be reduced in situ, include, without limitation, allylchloro[1,3-bis(2,6-di-i-propylphenyl)imidazol-2-ylidene]palladium(II), ([2S,3S]-bis[diphenylphosphino]butane)(η³-allyl)palladium(II) perchlorate, [S]-4-tert-butyl-2-(2-diphenylphosphanyl-phenyl)-4,5-dihydro-oxazole(η³-allyl)palladium(II) hexafluorophosphate (i.e., [Pd(S-tBu-PHOX)(allyl)]PF₆), and cyclopentadienyl(η³-allyl) palladium(II), with [Pd(s-tBu-PHOX)(allyl)]PF₆ and cyclopentadienyl(η³-allyl)palladium(II) being most preferred.

In certain embodiments, the transition metal is palladium. In certain embodiments, the transition metal catalyst is a dimer of a transition metal. Exemplary dimeric transition metal catalysts include Pd₂(dba)₃ and Pd₂(pmdba)₃. In certain preferred embodiments, the transition metal catalyst is Pd₂(dba)₃ or Pd₂(pmdba)₃. In embodiments of the method wherein the transition metal catalyst is a dimer, the amount of total transition metal present in the reaction is twice the amount of the transition metal catalytic complex.

Accordingly, when describing the amount of transition metal catalyst used in the methods of the invention, the following terminology applies. The amount of transition metal catalyst present in a reaction is alternatively referred to herein as “catalyst loading”. Catalyst loading may be expressed as a percentage that is calculated by dividing the moles of catalyst complex by the moles of the substrate present in a given reaction. Catalyst loading is alternatively expressed as a percentage that is calculated by dividing the moles of total transition metal (for example, palladium) by the moles of the substrate present in a given reaction. For example, in a reaction that uses 5 mol % dimeric catalyst (e.g, Pd₂(dba)₃), this amount of transition metal catalyst can be alternatively expressed as 10 mol % total transition metal (e.g., Pd(0)).

In certain embodiments, the transition metal catalyst is present under the conditions of the reaction from an amount of about 0.1 mol % to about 20 mol % total palladium relative to the substrate, which is the compound of formula (II). In certain embodiments, the catalyst loading is from about 1 mol % to about 15 mol % total palladium relative to the substrate. For example, in certain embodiments, the catalyst loading is about 1 mol %, about 2 mol %, about 3 mol %, about 5 mol %, about 6 mol %, about 8 mol %, about 9 mol %, about 10 mol %, about 11 mol %, about 12 mol %, or about 15 mol % total palladium.

### Ligands

One aspect of the invention relates to the enantioselectivity of the methods. Enantioselectivity results from the use of chiral ligands during the allylic alkylation reaction. Accordingly, in certain embodiments, the transition metal catalyst further comprises a chiral ligand. Without being bound by theory, the asymmetric environment that is created around the metal center by the presence of chiral ligands produces an enantioselective reaction. The chiral ligand forms a complex with the transition metal, thereby occupying one or more of the coordination sites on the metal and creating an asymmetric environment around the metal center. This complexation may or may not involve the displacement of achiral ligands already complexed to the metal. When displacement of one or more achiral ligands occurs, the displacement may proceed in a concerted fashion, i.e., with both the achiral ligand decomplexing from the metal and the chiral ligand complexing to the metal in a single step. Alternatively, the displacement may proceed in a stepwise fashion, i.e., with decomplexing of the achiral ligand and complexing of the chiral ligand occurring in distinct steps. Complexation of the chiral ligand to the transition metal may be allowed to occur in situ, i.e., by admixing the ligand and metal before adding the substrate. Alternatively, the ligand-metal complex can be formed separately, and the complex isolated before use in the alkylation reactions of the present invention.

Once coordinated to the transition metal center, the chiral ligand influences the orientation of other molecules as they interact with the transition metal catalyst. Coordination of the metal center with a π-allyl group and reaction of the substrate with the π-allyl-metal complex are dictated by the presence of the chiral ligand. The orientation of the reacting species determines the stereochemistry of the products.

Chiral ligands of the invention may be bidentate or monodentate or, alternatively, ligands with higher denticity (e.g., tridentate, tetradentate, etc.) can be used. Preferably, the ligand will be substantially enantiopure. By “enantiopure” is meant that only a single enantiomer is present. In many cases, substantially enantiopure ligands can be purchased from commercial sources, obtained by successive recrystallization of an enantioenriched substance, or by other suitable means for separating enantiomers.

Exemplary chiral ligands may be found in U.S. Pat. No. 7,235,698, the entirely of which is incorporated herein by reference. In certain embodiments, the chiral ligand is an enantioenriched phosphine ligand. In certain embodiments, the enantioenriched phosphine ligand is a P,N-ligand such as a phosphinooxazoline (PHOX) ligand. Preferred chiral ligands of the invention include the PHOX-type chiral ligands such as (R)-2-[2-(diphenylphosphino)phenyl]-4-isopropyl-2-oxazoline, (R)-2-[2-(diphenylphosphino)phenyl]-4-phenyl-2-oxazoline, (S)-2-[2-(diphenylphosphino)phenyl]-4-benzyl-2-oxazoline, (S)-2-[2-(diphenylphosphino)phenyl]-4-tert-butyl-2-oxazoline ((S)-t-BuPHOX) and (S)-2-(2-(bis(4-(Trifluoromethyl)phenyl)phosphino)-5-(trifluoromethyl)phenyl)-4-(tert-butyl)-4,5-dihydrooxazole ((S)—(CF₃)₃-t-BuPHOX). In preferred embodiments, the PHOX type chiral ligand is selected from (S)-t-BuPHOX and (S)—(CF₃)₃-t-BuPHOX). The ligand structures are depicted below.

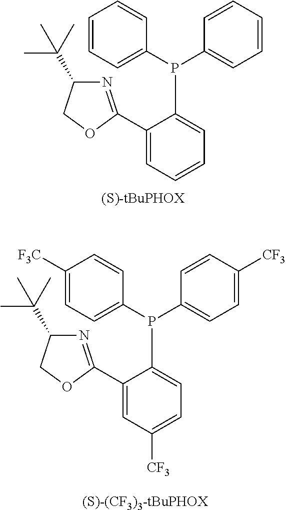

Generally, the chiral ligand is present in an amount in the range of about 0.75 equivalents to about 10 equivalents relative to the amount of total metal from the catalyst, preferably in the range of about 0.75 to about 5 equivalents relative to the amount of total metal from the catalyst, and most preferably in the range of about 0.75 to about 1.25, such as about 1.25 equivalents relative to the amount of total metal from the catalyst. Alternatively, the amount of the chiral ligand can be measured relative to the amount of the substrate.

In certain embodiments, the ligand is present under the conditions of the reaction from an amount of about 0.5 mol % to about 30 mol % relative to the substrate, which is the compound of formula (II). The amount of ligand present in the reaction is alternatively referred to herein as “ligand loading” and is expressed as a percentage that is calculated by dividing the moles of ligand by the moles of the substrate present in a given reaction. In certain embodiments, the ligand loading is from about 5 mol % to about 15 mol %. For example, in certain embodiments, the ligand loading is about about 5 mol %, about 6 mol %, about 7.5 mol %, about 9 mol %, about 10 mol %, about 11 mol %, about 12 mol %, about 12.5 mol %, about 13 mol %, about 14 mol %, or about 15 mol %. In certain embodiments, the ligand is in excess of the transition metal catalyst. In certain embodiments, the ligand loading is about 1.25 times the transition metal catalyst loading. In embodiments in which the transition metal catalyst is a dimer, the ligand loading is about 2.5 times the loading of the dimeric transition metal catalyst.

Where a chiral ligand is used, the reactions of the invention may enrich the stereocenter bearing R² in the product relative to the enrichment at this center, if any, of the starting material. In certain embodiments, the chiral ligand used in the methods of the invention yields a compound of formula (I) that is enantioenriched. The level of enantioenrichment of a compound may be expressed as enantiomeric excess (ee). The ee of a compound may be measured by dividing the difference in the fractions of the enantiomers by the sum of the fractions of the enantiomers. For example, if a compound is found to comprise 98% (S)-enantiomer, and 2% (R) enantiomer, then the ee of the compound is (98-2)/(98+2), or 96%. In certain embodiments, the compound of formula (I) has about 30% ee or greater, 40% ee or greater, 50% ee or greater, 60% ee or greater, 70% ee or greater, about 80% ee, about 85% ee, about 88% ee, about 90% ee, about 91% ee, about 92% ee, about 93% ee, about 94% ee, about 95% ee, about 96% ee, about 97% ee, about 98% ee, about 99% ee, or above about 99% ee, even where this % ee is greater than the % ee of the starting material, such as 0% ee (racemic). In certain embodiments, the compound of formula (I) is enantioenriched. In certain embodiments, the compound of formula (I) is enantiopure. In embodiments where the starting material has more than one stereocenter, reactions of the invention may enrich the stereocenter bearing R² relative to the enrichment at this center, if any, of the starting material, and substantially independently of the stereochemical disposition/enrichment of any other stereocenters of the molecule. For example, a product of the methods described herein may have 30% de or greater, 40% de or greater, 50% de or greater, 60% de or greater, 70% de or greater, 80% de or greater, 90% de or greater, 95% de or greater, or even 98% de or greater at the stereocenter of the product bearing R².

In certain embodiments, the invention also relates to methods that utilize an achiral ligand. Exemplary achiral ligands include triphenylphosphine, tricyclohexylphosphine, tri-(ortho-tolyl)phosphine, trimethylphosphite, and triphenylphosphite.

### Alkylation Conditions

In certain embodiments, the methods of the invention include treating a compound of formula (II) with a transition metal catalyst under alkylation conditions. In certain embodiments, alkylation conditions of the reaction include one or more organic solvents. In certain embodiments, organic solvents include aromatic or non-aromatic hydrocarbons, ethers, alkylacetates, nitriles, or combinations thereof. In certain embodiments, organic solvents include hexane, pentane, benzene, toluene, xylene, cyclic ethers such as optionally substituted tetrahydrofuran and dioxane, acyclic ethers such as dimethoxyethane, diethyl ether, methyl tertbutyl ether, and cyclopentyl methyl ether, acetonitrile, isobutyl acetate, ethyl acetate, isopropyl acetate, or combinations thereof. In certain preferred embodiments, the solvent is toluene, methyl tertbutyl ether, cyclopentyl methyl ether, 2-methyltetrahydrofuran, isobutyl acetate, ethyl acetate, or isopropyl acetate. In certain other preferred embodiments, the solvent is ethyl acetate.

In certain embodiments, alkylation conditions of the reaction include a reaction temperature. In certain embodiments, the reaction temperature is ambient temperature (about 20° C. to about 26° C.). In certain embodiments, the reaction temperature is higher than ambient temperature, such as, for example, about 30° C., about 35° C., about 40° C., about 45° C., about 50° C., about 55° C., or about 60° C. In certain embodiments, the reaction temperature is lower than ambient temperature, such as, for example, about 0° C.

In certain embodiments, instruments such as a microwave reactor may be used to accelerate the reaction time. Pressures range from atmospheric to pressures typically used in conjunction with supercritical fluids, with the preferred pressure being atmospheric.

### IV. Further Reactions of Products Generated by the Methods of the Invention

In certain embodiments, the invention includes methods for the preparation of a compound of formula (I),

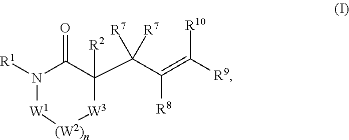

R¹ represents hydrogen or optionally substituted alkyl, cycloalkyl, (cycloalkyl)alkyl, aryl, aralkyl, heteroaryl, heteroaralkyl, alkenyl, alkynyl, —C(O)alkyl, —C(O)aryl, —C(O)aralkyl, —C(O)heteroaryl, —C(O)heteroaralkyl, —C(O)O(alkyl), —C(O)O(aryl), C(O)O(aralkyl), —C(O)O(heteroaryl), or —C(O)O(heteroaralkyl); or
R² represents hydrogen or substituted or unsubstituted alkyl, aralkyl, aryl, heteroaralkyl, heteroaryl, alkenyl, alkynyl, or halo;
R⁷, R⁸, R⁹, and R¹⁰ are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
W¹, W², and W³ are independently selected for each occurrence from CR¹¹R¹², O, and S;
R¹¹ and R¹² are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
wherein any occurrence of R¹¹ may optionally combine with any second occurrence of R¹¹ or an occurrence of R¹², along with the carbons to which they are attached, to form an optionally substituted 3-8 membered ring; and
n is an integer from 0-3;

wherein the compound of formula (I) is not

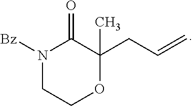

As demonstrated in Example 3, compounds of formula (I) can be elaborated into complex products through the application of further chemical transformations. In certain embodiments, these complex products have structural similarities to biologically- or pharmaceutically-relevant products, and can, therefore, play roles in medicinal chemistry discovery or serve as chiral building blocks to other molecules. In certain embodiments, these complex products are themselves biologically- or pharmaceutically-relevant products.

In an example embodiment, removal of the benzoyl protecting group, followed by reduction of the amide by LiAlH₄ can convert morpholinone 2c into N—H morpholine 7. In another example embodiment, acid treatment of 2h in methanol provides α-tertiary-hydroxy ester 8 in 71% yield without erosion of enantiopurity. In another exemplary functionalization reaction, α-quaternary δ-lactone 9 was synthesized from 4j in a good yield by zinc mediated reduction of the N—O bond followed by acid catalyzed cyclization.

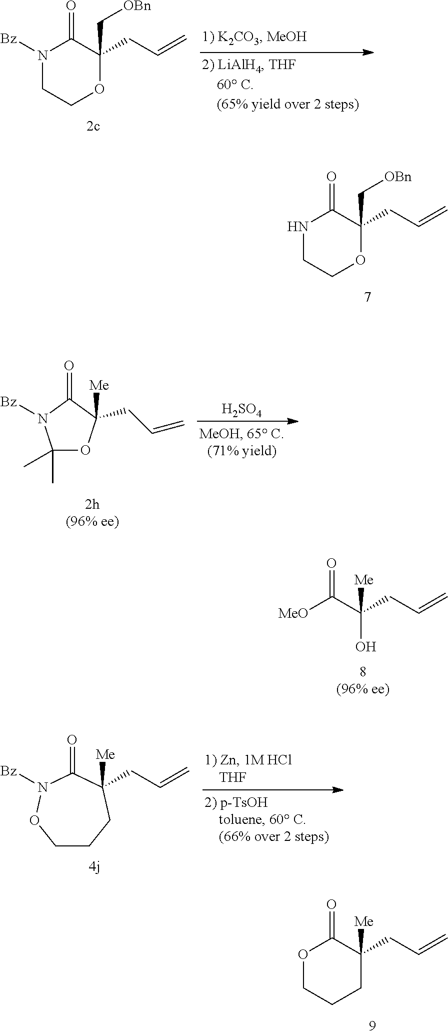

### EXEMPLIFICATION

The invention described generally herein will be more readily understood by reference to the following examples, which are included merely for purposes of illustration of certain aspects and embodiments of the present invention, and are not intended to limit the invention.

### Example 1

### Substrate Syntheses for Allylic Alkylation

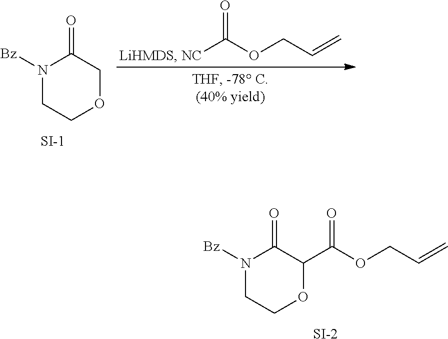

### Morpholinone SI-1

To a stirred solution of LiHMDS (3.89 g, 23.3 mmol, 2.2 equiv) in THF (40 mL) was added a solution of morpholinone SI-1 (2.17 g, 10.6 mmol, 1 equiv) in THF (30 mL) dropwise via syringe at −78° C. After stirring for 1 h, allyl cyanoformate (1.41 g, 12.7 mmol, 1.2 equiv) was added dropwise over 3 min at −78° C. After stirring at −78° C. for 3 h, the reaction mixture was poured into a stirred mixture of saturated aqueous ammonium chloride and diethyl ether, and the biphasic mixture was stirred at ambient temperature for 5 min and extracted with diethyl ether twice. The combined organic layers were dried over Na₂SO₄, filtered, and concentrated in vacuo. Flash column chromatography (SiO₂, 12→15% EtOAc in hexanes) afforded morpholinone SI-1 (1.23 g, 4.25 mmol, 40% yield) as a colorless oil. R_{f}=0.45 (33% EtOAc in hexanes); ¹H NMR (300 MHz, CDCl₃) δ 7.70-7.56 (m, 2H), 7.51 (m, 1H), 7.47-7.34 (m, 2H), 5.95 (ddt, J=17.2, 10.4, 5.9 Hz, 1H), 5.39 (m, 1H), 5.31 (m, 1H), 4.84 (s, 1H), 4.79-4.71 (m, 2H), 4.36 (m, 1H), 4.18-3.86 (m, 3H); ¹³C NMR (75 MHz, CDCl₃) δ 172.6, 166.5, 165.7, 134.6, 132.5, 131.0, 128.5, 128.3, 119.8, 77.4, 67.0, 62.5, 44.7; IR (Neat Film, NaCl) 2950, 1749, 1695, 1373, 1280, 1232, 1159, 1102, 1019, 988, 952, 729 cm⁻¹; HRMS (ESI-APCI+) m/z calc'd for C₁₅H₁₆NO₅ [M+H]⁺: 290.1023. found 290.1026.

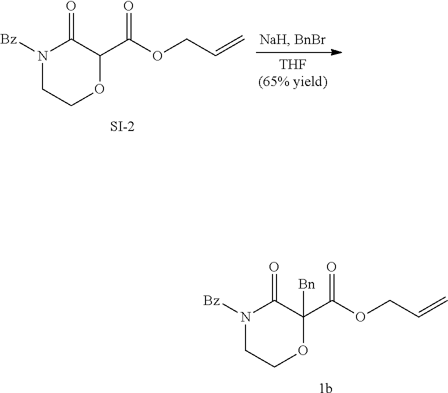

### Morpholinone 1b

To a stirred suspension of NaH (48.6 mg, 55 wt %, 1.11 mmol, 1.4 equiv) in THF (2.6 mL) was added a solution of morpholinone SI-2 (230 mg, 0.795 mmol, 1 equiv) in THF (2.0 mL) at 0° C. The reaction mixture was stirred at 0° C. for 30 min before the addition of benzyl bromide (0.170 mL, 1.43 mmol, 1.8 equiv). The reaction mixture was warmed to room temperature, stirred for 12 h and poured into a stirred mixture of saturated aqueous ammonium chloride and diethyl ether. The phases were separated, and the aqueous phase was extracted with diethyl ether twice. The combined organic layers were washed with saturated aqueous sodium bicarbonate and brine, dried over Na₂SO₄, filtered, and concentrated in vacuo. Flash column chromatography (SiO₂, 10→15% EtOAc in hexanes) afforded morpholinone 1b (196 mg, 0.517 mmol, 65% yield) as a colorless oil. R_{f}=0.45 (25% EtOAc in hexanes); ¹H NMR (500 MHz, CDCl₃) δ 7.56-7.43 (m, 3H), 7.42-7.34 (m, 2H), 7.34-7.22 (m, 5H), 5.98 (ddt, J=17.2, 10.4, 5.9 Hz, 1H), 5.42 (m, 1H), 5.35 (m, 1H), 4.81-4.73 (m, 2H), 4.28 (ddd, J=12.2, 10.7, 2.8 Hz, 1H), 4.03 (ddd, J=12.2, 3.8, 2.9 Hz, 1H), 3.75 (ddd, J=13.2, 2.9, 2.8 Hz, 1H), 3.43 (d, J=13.9 Hz, 1H), 3.31 (d, J=13.9 Hz, 1H), 3.29 (ddd, J=13.2, 10.7, 3.8 Hz, 1H); ¹³C NMR (126 MHz, CDCl₃) δ 172.6, 168.6, 167.6, 135.1, 134.6, 132.2, 131.3, 131.3, 128.5, 128.3, 128.2, 127.5, 119.7, 84.5, 66.9, 62.3, 44.4, 41.6; IR (Neat Film, NaCl) 2946, 1750, 1692, 1451, 1315, 1280, 1223, 1146, 1050, 1026, 945 cm⁻¹; HRMS (ESI-APCI+) m/z calc'd for C₂₂H₂₂NO₅ [M+H]⁺: 380.1492. found 380.1492.

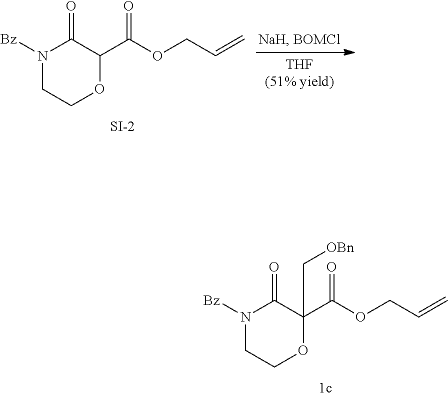

### Morpholinone 1c

To a stirred suspension of NaH (45.0 mg, 55 wt %, 1.03 mmol, 1.4 equiv) in THF (2.6 mL) was added a solution of morpholinone SI-2 (213 mg, 0.736 mmol, 1 equiv) in THF (2.6 mL) at 0° C. The reaction mixture was stirred at 0° C. for 30 min before the addition of benzyloxymethyl chloride (0.184 mL, 1.32 mmol, 1.8 equiv). The reaction mixture was warmed to room temperature, stirred for 5 h and poured into a stirred mixture of saturated aqueous ammonium chloride and diethyl ether. The phases were separated and the aqueous phase was extracted with diethyl ether twice. The combined organic layers were washed with saturated aqueous sodium bicarbonate and brine, dried over Na₂SO₄, filtered, and concentrated in vacuo. Flash column chromatography (SiO₂, 15% EtOAc in hexanes) afforded morpholinone 1c (155 mg, 0.379 mmol, 51% yield) as a white solid. R_{f}=0.48 (33% EtOAc in hexanes); m.p. 110.4-110.7° C.; ¹H NMR (500 MHz, CDCl₃) δ 7.68-7.61 (m, 2H), 7.52-7.43 (m, 1H), 7.40-7.24 (m, 7H), 5.91 (ddt, J=17.2, 10.4, 5.8 Hz, 1H), 5.35 (m, 1H), 5.28 (m, 1H), 4.72-4.65 (m, 2H), 4.66-4.60 (m, 2H), 4.38 (ddd, J=12.5, 9.7, 2.9 Hz, 1H), 4.30 (ddd, J=12.5, 3.7, 3.7 Hz, 1H), 4.17 (d, J=10.2 Hz, 1H), 4.10 (ddd, J=13.2, 3.7, 2.9 Hz, 1H), 3.94 (ddd, J=13.2, 9.7, 3.7 Hz, 1H), 3.92 (d, J=10.2 Hz, 1H); ¹³C NMR (126 MHz, CDCl₃) δ 172.7, 167.2, 167.0, 137.6, 134.9, 132.3, 131.1, 128.6, 128.6, 128.2, 128.0, 127.7, 119.6, 84.0, 74.3, 73.4, 66.8, 62.8, 44.7; IR (Neat Film, NaCl) 2941, 2873, 1747, 1690, 1449, 1371, 1318, 1280, 1231, 1160, 1073, 956, 727, 696 cm⁻¹; HRMS (ESI-APCI+) m/z calc'd for C₂₃H₂₄NO₆ [M+H]⁺: 410.1598. found 410.1598.

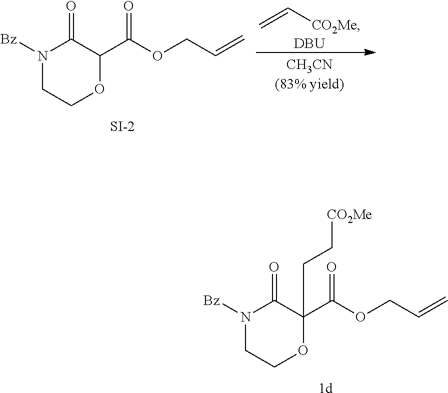

### Morpholinone 1d

To a stirred solution of morpholinone SI-2 (213 mg, 0.736 mmol, 1 equiv) in CH₃CN (4.5 mL) was added methyl acrylate (0.159 mL, 1.77 mmol, 2.0 equiv) and DBU (6.6 μL, 0.044 mmol, 0.05 equiv) at room temperature. After stirring at room temperature for 12 h, the reaction mixture was diluted with ethyl acetate (20 mL). The resulting mixture was washed with 1 M HCl, saturated aqueous sodium bicarbonate and brine, dried over Na₂SO₄, filtered, and concentrated in vacuo. Flash column chromatography (SiO₂, 20→25% EtOAc in hexanes) afforded morpholinone 1d (274 mg, 0.730 mmol, 83% yield) as a colorless oil. R_{f}=0.42 (33% EtOAc in hexanes); ¹H NMR (500 MHz, CDCl₃) δ 7.69-7.62 (m, 2H), 7.52 (m, 1H), 7.42-7.38 (m, 2H), 5.98 (ddt, J=17.2, 10.4, 6.0 Hz, 1H), 5.42 (m, 1H), 5.34 (m, 1H), 4.77-4.75 (m, 2H), 4.27 (ddd, J=12.3, 10.4, 3.2 Hz, 1H), 4.13 (ddd, J=12.3, 4.0, 3.1 Hz, 1H), 4.00 (ddd, J=13.2, 10.4, 4.0 Hz, 1H), 3.88 (ddd, J=13.2, 3.2, 3.1 Hz, 1H), 3.65 (s, 3H), 2.56-2.34 (m, 4H); ¹³C NMR (126 MHz, CDCl₃) δ 173.0, 173.0, 168.4, 167.9, 134.9, 132.4, 131.0, 128.5, 128.3, 120.1, 83.2, 67.1, 61.9, 51.9, 45.1, 30.8, 28.6; IR (Neat Film, NaCl) 2951, 1737, 1690, 1448, 1369, 1280, 1226, 1177, 1153, 1072, 944, 727, 694 cm⁻¹; HRMS (ESI-APCI+) m/z calc'd for C₁₉H₂₂NO₇ [M+H]⁺: 376.1391. found 376.1393.

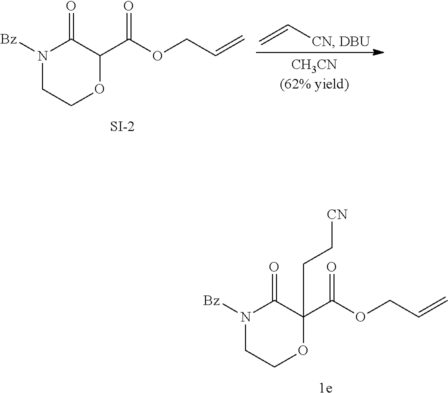

### Morpholinone 1e

To a stirred solution of morpholinone SI-2 (250 mg, 0.864 mmol, 1 equiv) in CH₃CN (4.3 mL) was added acrylonitrile (0.113 mL, 1.73 mmol, 2.0 equiv) and DBU (6.4 μL, 0.043 mmol, 0.05 equiv) at room temperature. After stirring at room temperature for 8 h, the reaction mixture was diluted with ethyl acetate (30 mL). The resulting mixture was washed with 1 M HCl, saturated aqueous sodium bicarbonate and brine, dried over Na₂SO₄, filtered, and concentrated in vacuo. Flash column chromatography (SiO₂, 20→25% EtOAc in hexanes) afforded morpholinone 1e (182 mg, 0.532 mmol, 62% yield) as a white solid. R_{f}=0.41 (33% EtOAc in hexanes); ¹H NMR (500 MHz, CDCl₃) δ 7.67-7.64 (m, 2H), 7.54 (m, 1H), 7.44-7.39 (m, 2H), 5.99 (ddt, J=17.1, 10.4, 6.0 Hz, 1H), 5.46-5.36 (m, 2H), 4.81-4.78 (m, 2H), 4.32 (ddd, J=12.4, 10.4, 3.1 Hz, 1H), 4.18 (ddd, J=12.4, 4.0, 3.1 Hz, 1H), 4.04 (ddd, J=13.4, 10.4, 4.0 Hz, 1H), 3.89 (ddd, J=13.4, 3.1, 3.1 Hz, 1H), 2.60-2.36 (m, 4H); ¹³C NMR (126 MHz, CDCl₃) δ 172.8, 167.7, 167.3, 134.6, 132.7, 130.7, 128.5, 128.4, 120.6, 118.8, 76.9, 67.5, 62.0, 45.1, 31.2, 12.1; IR (Neat Film, NaCl) 3062, 2950, 2894, 2248, 1746, 1692, 1600, 1462, 1449, 1372, 1280, 1221, 1155, 1070, 943, 796, 727, 694 cm⁻¹; HRMS (ESI-APCI+) m/z calc'd for C₁₈H₁₉N₂O₅ [M+H]⁺: 343.1288. found 343.1290.

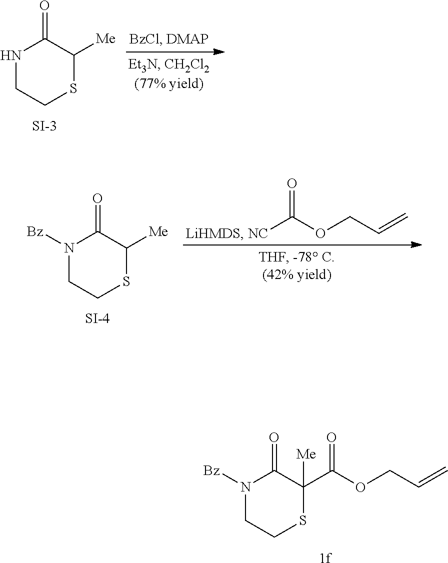

### Thiomorpholinone SI-4

To a stirred solution of morpholinone SI-3 (1.02 g, 7.77 mmol, 1 equiv), DMAP (47.4 mg, 0.389 mmol, 0.05 equiv) and Et₃N (2.48 mL, 17.9 mmol, 2.3 equiv) in CH₂Cl₂ (24 mL) was added benzoyl chloride (0.994 mL, 8.55 mmol, 1.1 equiv) at 0° C. The reaction mixture was warmed to room temperature gradually and stirred for 20 h. After full consumption of the starting material as indicated by TLC analysis, the reaction mixture was diluted with ethyl acetate (30 mL) and washed with 1 M HCl, saturated aqueous sodium bicarbonate and brine. The organic layers were dried over Na₂SO₄, filtered, and concentrated in vacuo. Flash column chromatography (SiO₂, 10→15% EtOAc in hexanes) afforded thiomorpholinone SI-4 (1.40 g, 5.95 mmol, 77% yield) as a yellow solid. R_{f}=0.41 (25% EtOAc in hexanes); m.p. 94.0-94.7° C.; ¹H NMR (500 MHz, CDCl₃) δ 7.60-7.57 (m, 2H), 7.51 (m, 1H), 7.44-7.39 (m, 2H), 4.81 (ddd, J=14.3, 5.0, 3.3 Hz, 1H), 3.86 (q, J=6.7 Hz, 1H), 3.72 (ddd, J=14.3, 11.8, 4.1 Hz, 1H), 3.19-3.06 (m, 2H), 1.38 (d, J=6.6 Hz, 3H); ¹³C NMR (126 MHz, CDCl₃) δ 173.4, 172.6, 136.0, 132.0, 128.3, 128.2, 43.9, 37.2, 27.2, 14.4; IR (Neat Film, NaCl) 2932, 1683, 1373, 1318, 1279, 1130, 991, 878 cm⁻¹; HRMS (ESI-APCI+) m/z calc'd for C₁₂H₁₄NO₂S [M+H]⁺: 236.0740. found 236.0737.

Thiomorpholinone 1f.

To a stirred solution of LiHMDS (277 mg, 1.66 mmol, 1.3 equiv) in THF (5 mL) was added a solution of thiomorpholinone SI-4 (300 mg, 1.27 mmol, 1 equiv) in THF (3 mL) dropwise via syringe at −78° C. After stirring for 1 h, allyl cyanoformate (169 mg, 1.52 mmol, 1.2 equiv) was added dropwise over 3 min at −78° C. After stirring at −78° C. for 3 h, the reaction mixture was poured into a stirred mixture of saturated aqueous ammonium chloride and diethyl ether, and the biphasic mixture was stirred at ambient temperature for 5 min and extracted with diethyl ether twice. The combined organic layers were dried over Na₂SO₄, filtered, and concentrated in vacuo. Flash column chromatography (SiO₂, 5→8% EtOAc in hexanes) afforded morpholinone if (172 mg, 0.539 mmol, 42% yield) as a yellow oil. R_{f}=0.48 (25% EtOAc in hexanes); ¹H NMR (500 MHz, CDCl₃) δ 7.74-7.68 (m, 2H), 7.50 (m, 1H), 7.42-7.38 (m, 2H), 5.98 (m, 1H), 5.44 (m, 1H), 5.35 (m, 1H), 4.77 (br d, J=5.9 Hz, 2H), 4.55 (m, 1H), 3.84 (ddd, J=14.3, 8.5, 3.6 Hz, 1H), 3.27 (ddd, J=13.0, 6.9, 3.6 Hz, 1H), 3.00 (ddd, J=13.0, 8.5, 4.5 Hz, 1H), 1.66 (s, 3H); ¹³C NMR (126 MHz, CDCl₃) δ 174.3, 171.0, 169.1, 135.6, 132.2, 131.1, 128.3, 128.2, 120.0, 67.2, 52.8, 46.8, 26.5, 22.2; IR (Neat Film, NaCl) 2939, 1743, 1681, 1691, 1449, 1378, 1314, 1265, 1220, 1109, 990, 884 cm⁻¹; HRMS (ESI-APCI+) m/z calc'd for C₁₆H₁₈NO₄S [M+H]⁺: 320.0951. found 320.0957.

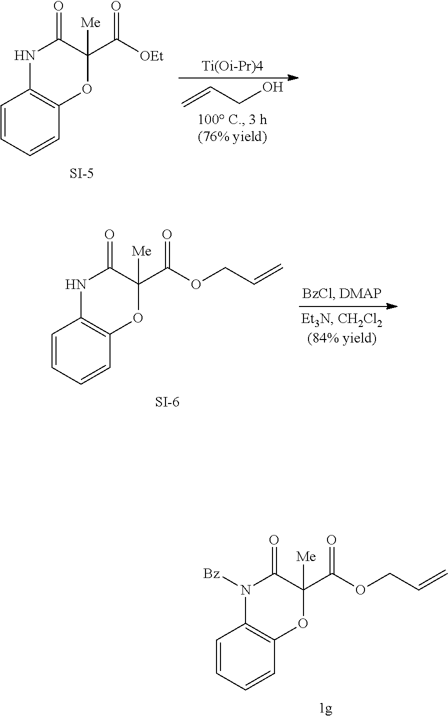

### Benzomorpholinone SI-6

To a solution of benzomorpholinone SI-5 (1.02 g, 7.77 mmol, 1 equiv) in allyl alcohol (3.0 mL) was added Ti(Oi-Pr)₄ (0.076 mL, 0.260 mmol, 0.2 equiv) at room temperature. After stirring at 100° C. for 3 h, the reaction mixture was diluted with ethyl acetate (50 mL) and washed with 1 M HCl, saturated aqueous sodium bicarbonate and brine. The organic layers were dried over Na₂SO₄, filtered, and concentrated in vacuo. Flash column chromatography (SiO₂, 7→15% EtOAc in hexanes) afforded thiomorpholinone SI-6 (239 mg, 0.976 mmol, 76% yield) as a white solid. R_{f}=0.40 (33% EtOAc in hexanes); m.p. 82.3-84.1° C.; ¹H NMR (500 MHz, CDCl₃) δ 7.09-6.94 (m, 3H), 6.79 (m, 1H), 5.76 (m, 1H), 5.23-5.13 (m, 2H), 4.66-4.54 (m, 2H), 1.88 (s, 3H); ¹³C NMR (126 MHz, CDCl₃) δ 168.3, 164.8, 143.0, 131.1, 126.2, 124.6, 123.2, 118.8, 117.5, 116.0, 81.3, 66.7, 20.8; IR (Neat Film, NaCl) 3235, 1744, 1698, 1614, 1502, 1379, 1232, 1123, 968, 751 cm⁻¹; HRMS (ESI-APCI+) m/z calc'd for C₁₃H₁₄NO₄ [M+H]⁺: 248.0917. found 248.0907.

Benzomorpholinone 1g.

To a stirred solution of benzomorpholinone SI-6 (150 mg, 0.607 mmol, 1 equiv), DMAP (7.4 mg, 0.061 mmol, 0.10 equiv) and Et₃N (0.127 mL, 0.911 mmol, 1.5 equiv) in CH₂Cl₂ (3 mL) was added benzoyl chloride (0.084 mL, 0.728 mmol, 1.2 equiv) at room temperature. After full consumption of the starting material as indicated by TLC analysis, the reaction mixture was diluted with diethyl ether (30 mL) (30 mL) and washed with 1 M HCl, saturated aqueous sodium bicarbonate and brine. The organic layers were dried over Na₂SO₄, filtered, and concentrated in vacuo. Flash column chromatography (SiO₂, 10→13% EtOAc in hexanes) afforded thiomorpholinone 1g (180 mg, 0.512 mmol, 84% yield) as a colorless oil. R_{f}=0.19 (10% EtOAc in hexanes); ¹H NMR (500 MHz, CDCl₃) δ 8.01-7.98 (m, 2H), 7.64 (ddt, J=7.8, 7.1, 1.3 Hz, 1H), 7.51-7.47 (m, 2H), 7.18 (m, 1H), 7.11 (ddd, J=8.1, 7.3, 1.5 Hz, 1H), 6.99 (ddd, J=8.1, 7.3, 1.5 Hz, 1H), 6.90 (ddd, J=8.1, 1.5, 0.4 Hz, 1H), 5.78 (ddt, J=17.2, 10.5, 5.8 Hz, 1H), 5.27-5.21 (m, 2H), 4.65-4.63 (m, 2H), 1.87 (s, 3H); ¹³C NMR (126 MHz, CDCl₃) δ 171.8, 168.5, 163.8, 144.3, 134.6, 133.6, 130.9, 130.3, 129.1, 127.2, 125.8, 123.7, 119.6, 118.6, 118.3, 81.6, 66.9, 20.5; IR (Neat Film, NaCl) 3070, 1726, 1708, 1496, 1338, 1282, 1240, 1123 cm⁻¹; HRMS (ESI-APCI+) m/z calc'd for C₂₀H₁₈NO₅ [M+H]⁺: 352.1179. found 352.1163.

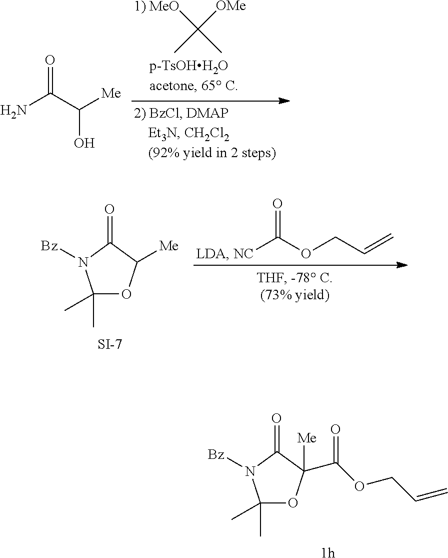

Oxazolidinone SI-7.

To a suspension of lactamide (2.50 g, 28.1 mmol, 1 equiv) and 2,2-dimethoxypropane (8.76 mL, 84.2 mmol, 3.0 equiv) in acetone (30 mL) was added p-toluenesulfonic acid monohydrate (53.0 mg, 0.280 mmol, 0.01 equiv) at room temperature. The reaction mixture was warmed to 65° C. and stirred for 2 h. After full consumption of the starting material as indicated by TLC analysis, the reaction mixture was queched with Et₃N and concentrated in vacuo. The residue was used for the next reaction without further purification.

To a stirred solution of the crude acetonide, DMAP (189 mg, 1.54 mmol, 0.05 equiv) and Et₃N (5.87 mL, 42.2 mmol, 1.5 equiv) in CH₂Cl₂ (60 mL) was added benzoyl chloride (3.57 mL, 30.9 mmol, 1.1 equiv) at 0° C. After full consumption of the starting material as indicated by TLC analysis, the reaction mixture was diluted with diethyl ether (30 mL) (30 mL) and washed with 1 M HCl, saturated aqueous sodium bicarbonate and brine. The organic layers were dried over Na₂SO₄, filtered, and concentrated in vacuo. Flash column chromatography (SiO₂, 10→15% EtOAc in hexanes) afforded oxazolidinone SI-7 (6.02 g, 25.8 mmol, 92% yield in 2 steps) as a white solid. R_{f}=0.41 (15% EtOAc in hexanes); m.p. 66.7-67.1° C.; ¹H NMR (500 MHz, CDCl₃) δ 7.61-7.52 (m, 3H), 7.47-7.41 (m, 2H), 4.42 (q, J=6.7 Hz, 1H), 1.78 (s, 3H), 1.77 (s, 3H), 1.51 (d, J=6.7 Hz, 3H); ¹³C NMR (126 MHz, CDCl₃) δ 173.3, 169.7, 134.4, 132.7, 129.1, 128.3, 95.8, 72.0, 26.8, 25.2, 17.5; IR (Neat Film, NaCl) 1756, 1684, 1309, 1292, 1282, 1156, 835 cm⁻¹.

Oxazolidinone 1h.

To a stirred solution of N,N-diisopropylamine (0.830 mL, 5.93 mmol, 1.3 equiv) in THF (15 mL) was added n-BuLi (2.83 mL, 2.3 M solution in hexanes, 5.47 mmol, 1.2 equiv) dropwise via syringe at −78° C. After stirring at 0° C. for 20 min, a solution of oxazolidinone SI-7 (1.00 g, 4.56 mmol, 1 equiv) in THF (10 mL) was added dropwise over 10 min at −78° C. After stirring at −78° C. for 30 min, allyl cyanoformate (659 mg, 5.93 mmol, 1.3 equiv) was added dropwise over 3 min at −78° C. After stirring at −78° C. for 2 h, the reaction mixture was poured into a stirred mixture of saturated aqueous ammonium chloride and diethyl ether, and the biphasic mixture was stirred at ambient temperature for 5 min and extracted with diethyl ether twice. The combined organic layers were dried over Na₂SO₄, filtered, and concentrated in vacuo. Flash column chromatography (SiO₂, 5→7% EtOAc in hexanes) afforded morpholinone 1h (1.06 g, 3.34 mmol, 73% yield) as a white solid. R_{f}=0.42 (15% EtOAc in hexanes); m.p. 95.0-95.7° C.; ¹H NMR (500 MHz, CDCl₃) δ 7.62-7.53 (m, 3H), 7.44-7.39 (m, 2H), 5.95 (ddt, J=17.2, 10.4, 5.8 Hz, 1H), 5.40 (m, 1H), 5.32 (m, 1H), 4.78-4.69 (m, 2H), 1.84 (s, 3H), 1.82 (s, 3H), 1.70 (s, 3H); ¹³C NMR (126 MHz, CDCl₃) δ 169.4, 169.2, 168.5, 134.2, 132.7, 131.2, 128.9, 128.3, 119.7, 97.0, 81.4, 67.0, 29.3, 26.7, 21.9; IR (Neat Film, NaCl) 2991, 1762, 1736, 1690, 1373, 1323, 1279, 1241, 1178, 1127, 994, 951, 834 cm⁻¹; HRMS (ESI-APCI+) m/z calc'd for C₁₇H₂₀NO₅ [M+H]⁺: 318.1336. found 318.1333.

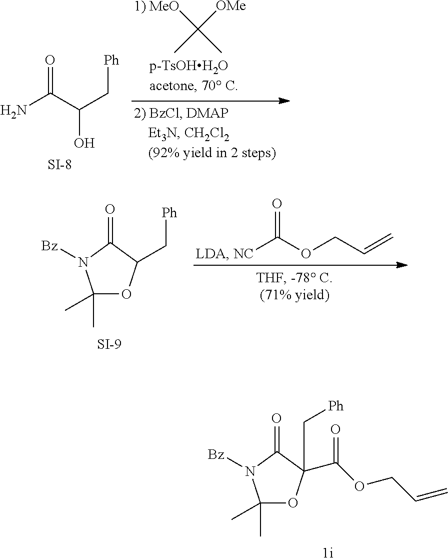

### Oxazolidinone SI-9

To a solution of amide SI-8 (800 mg, 4.48 mmol, 1 equiv) and 2,2-dimethoxypropane (1.78 mL, 14.5 mmol, 3.0 equiv) in acetone (10 mL) was added p-toluenesulfonic acid monohydrate (9.2 mg, 0.048 mmol, 0.01 equiv) at room temperature. The reaction mixture was warmed to 70° C. and stirred for 12 h. After full consumption of the starting material as indicated by TLC analysis, the reaction mixture was queched with Et₃N and concentrated in vacuo. The residue was used for the next reaction without further purification.

To a stirred solution of the crude acetonide, DMAP (29.6 mg, 0.242 mmol, 0.05 equiv) and Et₃N (1.10 mL, 7.26 mmol, 1.5 equiv) in CH₂Cl₂ (20 mL) was added benzoyl chloride (0.615 mL, 5.32 mmol, 1.1 equiv) at 0° C. After full consumption of the starting material as indicated by TLC analysis, the reaction mixture was diluted with diethyl ether (30 mL) and washed with 1 M HCl, saturated aqueous sodium bicarbonate and brine. The organic layers were dried over Na₂SO₄, filtered, and concentrated in vacuo. Flash column chromatography (SiO₂, 7→10% EtOAc in hexanes) afforded oxazolidinone SI-9 (1.47 g, 4.78 mmol, 98% yield in 2 steps) as a colorless oil. R_{f}=0.42 (10% EtOAc in hexanes); ¹H NMR (500 MHz, CDCl₃) δ 7.50 (m, 1H), 7.41-7.29 (m, 7H), 7.11-7.07 (m, 2H), 4.66 (dd, J=4.9, 3.8 Hz, 1H), 3.23 (dd, J=14.5, 3.8 Hz, 1H), 3.11 (dd, J=14.5, 4.9 Hz, 1H), 1.76 (s, 3H), 1.60 (s, 3H); ¹³C NMR (126 MHz, CDCl₃) δ 171.8, 169.8, 134.3, 132.7, 131.6, 130.9, 129.1, 128.3, 128.1, 127.2, 96.0, 76.3, 37.3, 26.1, 25.9; IR (Neat Film, NaCl) 1755, 1688, 1382, 1304, 1284, 1242, 1210, 1138 cm⁻¹; HRMS (ESI-APCI+) m/z calc'd for C₁₉H₂₀NO₃ [M+H]⁺: 310.1438. found 310.1426.

Oxazolidinone 1i.

To a stirred solution of LiHMDS (232 mg, 1.39 mmol, 1.4 equiv) in THF (3 mL) was added a solution of oxazolidinone SI-9 (307 mg, 0.992 mmol, 1 equiv) in THF (2 mL) dropwise via syringe at −78° C. After stirring for 1 h, allyl cyanoformate (132 mg, 1.19 mmol, 1.2 equiv) was added dropwise over 3 min at −78° C. After stirring at −78° C. for 2.5 h, the reaction mixture was poured into a stirred mixture of saturated aqueous ammonium chloride and diethyl ether, and the biphasic mixture was stirred at ambient temperature for 5 min and extracted with diethyl ether twice. The combined organic layers were dried over Na₂SO₄, filtered, and concentrated in vacuo. Flash column chromatography (SiO₂, 5→7% EtOAc in hexanes) afforded oxazolidinone 1i (279 mg, 0.709 mmol, 71% yield) as a colorless oil. R_{f}=0.42 (10% EtOAc in hexanes); ¹H NMR (500 MHz, CDCl₃) δ 7.52 (m, 1H), 7.39-7.31 (m, 7H), 7.16-7.13 (m, 2H), 5.95 (ddt, J=17.2, 10.4, 5.9 Hz, 1H), 5.40 (m, 1H), 5.30 (m, 1H), 4.79-4.70 (m, 2H), 3.41 (d, J=14.2 Hz, 1H), 3.34 (d, J=14.2 Hz, 1H), 1.81 (s, 3H), 1.38 (s, 3H); ¹³C NMR (126 MHz, CDCl₃) δ 169.2, 168.4, 167.2, 134.1, 134.0, 132.6, 131.9, 131.1, 128.9, 128.3, 128.1, 127.8, 119.9, 97.2, 85.2, 67.1, 40.7, 27.7, 27.1; IR (Neat Film, NaCl) 1754, 1692, 1309, 1278, 1235, 1156, 1039 cm⁻¹; HRMS (ESI-APCI+) m/z calc'd for C₂₃H₂₄NO₅ [M+H]⁺: 394.1649. found 394.1647.

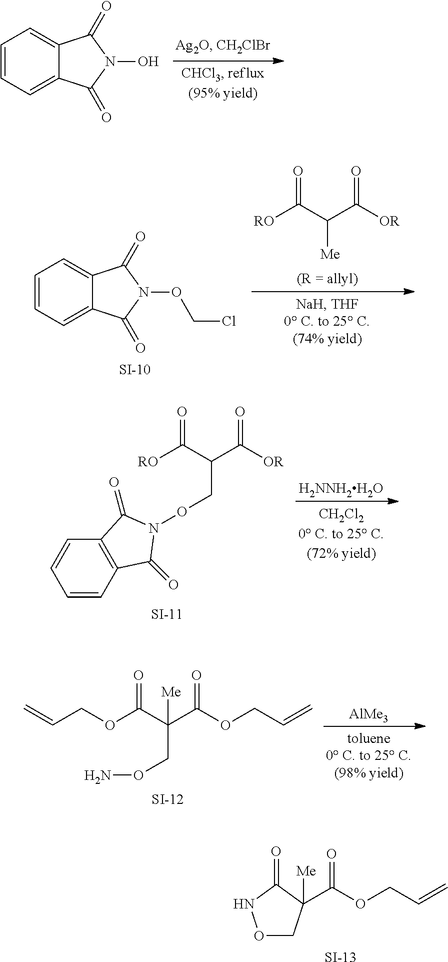

### N-Chloromethoxyphthalimide (SI-10)

N-Hydroxyphthalimide (1.06 g, 6.47 mmol, 3.0 equiv) and CH₂ClBr (4.2 mL, 64.7 mmol, 10 equiv) in CHCl₃ (50 mL) were heated at reflux for 30 min, then Ag₂O (0.50 g, 2.16 mmol, 1 equiv) was added with vigorous stirring. The suspension was stirred at 75° C. for 18 h under the dark and the reaction mixture was filtrated. The filtrate was concentrated in vacuo. Flash column chromatography (SiO₂, 10→20% EtOAc in hexanes) afforded N-chloromethoxyphthalimide (SI-10) (433 mg, 2.05 mmol, 95% yield) as a white solid. R_{f}=0.46 (33% EtOAc in hexanes); m.p. 112.9-114.0° C.; ¹H NMR (300 MHz, CDCl₃) δ 7.95-7.75 (m, 4H), 5.88 (s, 2H); ¹³C NMR (126 MHz, CDCl₃) δ 162.9, 135.0, 129.0, 124.1, 83.8; IR (Neat Film, NaCl) 1724, 1126, 1018, 1000, 874 cm⁻¹.

Malonate SI-11.

To a stirred suspension of NaH (397 mg, 60 wt %, 9.92 mmol, 1.5 equiv) in THF (20 mL) was added diallyl 2-methylmalonate (1.97 g, 9.92 mmol, 1.5 equiv) at 0° C. The reaction mixture was stirred at 0° C. for 15 min, and then a solution of N-chloromethoxyphthalimide (SI-10) (1.40 g, 6.62 mmol, 1 equiv) was added dropwise over 15 min at 0° C. The reaction mixture was warmed to room temperature, stirred for 8 h and poured into a stirred mixture of 1 M HCl and diethyl ether. The phases were separated and the aqueous phase was extracted with ethyl acetate twice. The combined organic layers were washed with saturated aqueous sodium bicarbonate and brine, dried over Na₂SO₄, filtered, and concentrated in vacuo. Flash column chromatography (SiO₂, 15→20% EtOAc in hexanes) malonate SI-11 (1.82 g, 4.87 mmol, 74% yield) as a colorless oil. R_{f}⁼0.22 (20% EtOAc in hexanes); ¹H NMR (300 MHz, CDCl₃) δ 7.84-7.72 (m, 4H), 5.98-5.84 (m, 2H), 5.36-5.20 (m, 4H), 4.72-4.65 (m, 4H), 4.62 (s, 2H), 1.78 (s, 3H); ¹³C NMR (126 MHz, CDCl₃) δ 169.1, 163.0, 134.6, 131.6, 129.1, 123.7, 118.8, 79.8, 66.5, 54.5, 18.1; IR (Neat Film, NaCl) 2946, 1792, 1736, 1467, 1379, 1287, 1249, 1188, 1125, 1021, 1002 cm⁻¹; HRMS (ESI-APCI+) m/z calc'd for C₁₉H₂₀NO₇ [M+H]⁺: 374.1234. found 374.1228.

Alkoxyamine SI-12.

To a stirred solution of malonate SI-11 (1.82 g, 4.87 mmol, 1 equiv) in CH₂Cl₂ (25 mL) was added hydrazine monohydrate (0.260 mL, 5.36 mmol, 1.1 equiv) at 0° C. The reaction mixture was warmed to room temperature, stirred for 20 h and filtered. The filtrate was concentrated in vacuo. Flash column chromatography (SiO₂, 15→20% EtOAc in hexanes) afforded alkoxyamine SI-12 (850 mg, 3.49 mmol, 72% yield) as a colorless oil. R_{f}=0.24 (25% EtOAc in hexanes); ¹H NMR (500 MHz, CDCl₃) δ 5.92-5.84 (m, 2H), 5.34-5.29 (m, 2H), 5.24-5.21 (m, 2H), 4.64-4.62 (m, 6H), 4.12 (s, 2H), 1.53 (s, 3H); ¹³C NMR (126 MHz, CDCl₃) δ 170.4, 131.8, 118.4, 78.4, 66.0, 54.6, 18.3; IR (Neat Film, NaCl) 2943, 1732, 1454, 1248, 1213, 1120, 1020, 988, 935 cm⁻¹; HRMS (ESI-APCI+) m/z calc'd for C₁₁H₁₈NO₅ [M+H]⁺: 244.1179. found 244.1175.

Isoxazolidinone SI-13.

To a stirred solution of alkoxyamine SI-12 (850 mg, 3.49 mmol, 1 equiv) in toluene (35 mL) was added trimethylaluminum (3.5 mL, 2.0 M solution in toluene, 6.98 mmol, 2.0 equiv) dropwise at 0° C. The reaction mixture was warmed to room temperature, stirred for 6 h, and poured into a stirred mixture of 1 M HCl and diethyl ether. The phases were separated and the aqueous phase was extracted with ethyl acetate twice. The combined organic layers were washed with brine, dried over Na₂SO₄, filtered, and concentrated in vacuo. Flash column chromatography (SiO₂, 20→40% EtOAc in hexanes) afforded isoxazolidinone SI-13 (633 mg, 3.42 mmol, 98% yield) as a colorless oil. R_{f}=0.22 (25% EtOAc in hexanes); ¹H NMR (500 MHz, CDCl₃) δ 5.91 (m, 1H), 5.35 (m, 1H), 5.26 (m, 1H), 4.81 (d, J=8.8 Hz, 1H), 4.71-4.69 (m, 2H), 4.17 (d, J=8.8 Hz, 1H), 1.55 (s, 3H); ¹³C NMR (126 MHz, CDCl₃) δ 172.7, 169.5, 131.4, 118.9, 78.3, 66.7, 53.7, 17.7; IR (Neat Film, NaCl) 3182, 3087, 1739, 1704, 1453, 1275, 1215, 1137, 1037, 934 cm⁻¹; HRMS (ESI-APCI+) m/z calc'd for C₈H₁₂NO₄ [M+H]⁺: 186.0761. found 186.0755.

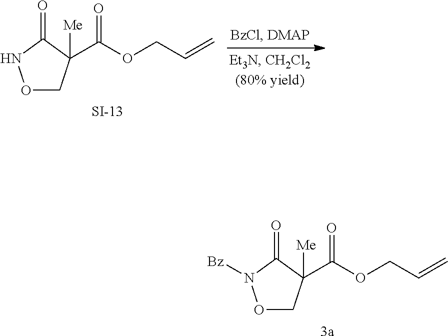

### Isoxazolidinone 3a

To a stirred solution of isoxazolidinone SI-13 (68.0 mg, 0.367 mmol, 1 equiv), DMAP (22.4 mg, 0.184 mmol, 0.50 equiv) and Et₃N (0.127 mL, 0.911 mmol, 2.5 equiv) in CH₂Cl₂ (2 mL) was added benzoyl chloride (0.064 mL, 0.551 mmol, 1.5 equiv) at room temperature. After full consumption of the starting material as indicated by TLC analysis, the reaction mixture was diluted with diethyl ether (30 mL) and washed with 1 M HCl, saturated aqueous sodium bicarbonate and brine. The organic layers were dried over Na₂SO₄, filtered, and concentrated in vacuo. Flash column chromatography (SiO₂, 15→20% EtOAc in hexanes) afforded isoxazolidinone 3a (82.5 mg, 0.295 mmol, 88% yield) as a colorless oil. R_{f}=0.25 (20% EtOAc in hexanes); ¹H NMR (500 MHz, CDCl₃) δ 7.76-7.73 (m, 2H), 7.59 (m, 1H), 7.48-7.43 (m, 2H), 5.92 (ddt, J=17.2, 10.5, 5.7 Hz, 1H), 5.39-5.28 (m, 2H), 4.92 (d, J=9.0 Hz, 1H), 4.74-4.71 (m, 2H), 4.28 (d, J=9.0 Hz, 1H), 1.61 (s, 3H); ¹³C NMR (126 MHz, CDCl₃) δ 168.6, 167.6, 163.8, 133.3, 131.8, 131.1, 129.9, 128.2, 119.4, 76.5, 67.1, 55.2, 17.5; IR (Neat Film, NaCl) 2942, 1769, 1741, 1703, 1450, 1273, 1138, 996 cm⁻¹; HRMS (ESI-APCI+) m/z calc'd for C₁₅H₁₆NO₅ [M+H]⁺: 290.1023. found 290.1013.

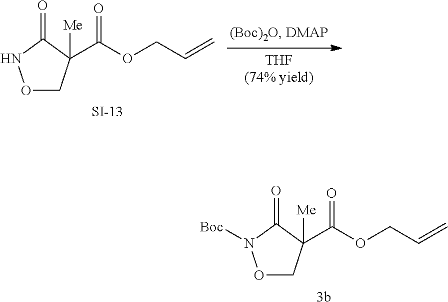

### Isoxazolidinone 3b

To a stirred solution of isoxazolidinone SI-13 (150 mg, 0.810 mmol, 1 equiv) and DMAP (19.8 mg, 0.162 mmol, 0.20 equiv) in THF (4 mL) was added (Boc)₂O (229 mg, 1.05 mmol, 1.3 equiv) at room temperature. After full consumption of the starting material as indicated by TLC analysis, the reaction mixture was concentrated in vacuo. Flash column chromatography (SiO₂, 10→15% EtOAc in hexanes) afforded isoxazolidinone 3b (170 mg, 0.596 mmol, 74% yield) as a colorless oil. R_{f}=0.35 (25% EtOAc in hexanes); ¹H NMR (500 MHz, CDCl₃) δ 5.90 (m, 1H), 5.34 (m, 1H), 5.26 (m, 1H), 4.79 (d, J=8.9 Hz, 1H), 4.70-4.68 (m, 2H), 4.14 (d, J=8.9 Hz, 1H), 1.58 (s, 3H), 1.57 (s, 9H); ¹³C NMR (126 MHz, CDCl₃) δ 168.7, 166.0, 146.2, 131.2, 119.1, 85.7, 76.1, 66.9, 55.0, 28.1, 17.6; IR (Neat Film, NaCl) 2984, 1791, 1748, 1458, 1371, 1291, 1157, 1107, 987, 946, 842, 752 cm⁻¹; HRMS (ESI-APCI+) m/z calc'd for C₁₃H₂₃N₂O₆ [M+NH₄]⁺: 303.1551. found 303.1539.

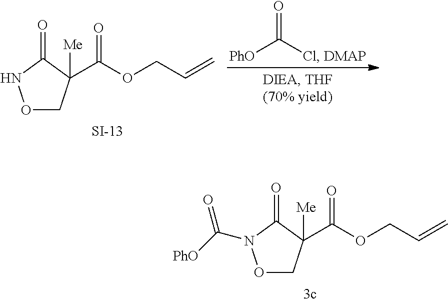

### Isoxazolidinone 3c

To a stirred solution of isoxazolidinone SI-13 (150 mg, 0.810 mmol, 1 equiv), DMAP (10.0 mg, 0.081 mmol, 0.10 equiv) and DIEA (0.353 mL, 2.03 mmol, 2.5 equiv) in CH₂Cl₂ (4 mL) was added phenyl chloroformate (0.132 mL, 1.05 mmol, 1.3 equiv) at 0° C. After full consumption of the starting material as indicated by TLC analysis, the reaction mixture was diluted with diethyl ether (30 mL) and washed with 1 M HCl, saturated aqueous sodium bicarbonate and brine. The organic layers were dried over Na₂SO₄, filtered, and concentrated in vacuo. Flash column chromatography (SiO₂, 15→20% EtOAc in hexanes) afforded isoxazolidinone 3a (82.5 mg, 0.295 mmol, 88% yield) as a colorless oil. R_{f}=0.22 (25% EtOAc in hexanes); ¹H NMR (500 MHz, CDCl₃) δ 7.44-7.39 (m, 2H), 7.31-7.22 (m, 3H), 5.95 (m, 1H), 5.38 (m, 1H), 5.30 (m, 1H), 4.91 (d, J=9.0 Hz, 1H), 4.75-4.73 (m, 2H), 4.28 (d, J=9.0 Hz, 1H), 1.65 (s, 3H); ¹³C NMR (126 MHz, CDCl₃) δ 168.4, 166.1, 150.1, 146.0, 131.1, 129.7, 126.7, 121.3, 119.4, 76.7, 67.1, 54.9, 17.6; IR (Neat Film, NaCl) 1802, 1761, 1315, 1220, 1192, 1138, 980, 936 cm⁻¹; HRMS (ESI-APCI+) m/z calc'd for C₁₅H₁₆NO₆ [M+H]⁺: 306.0972. found 306.0959.

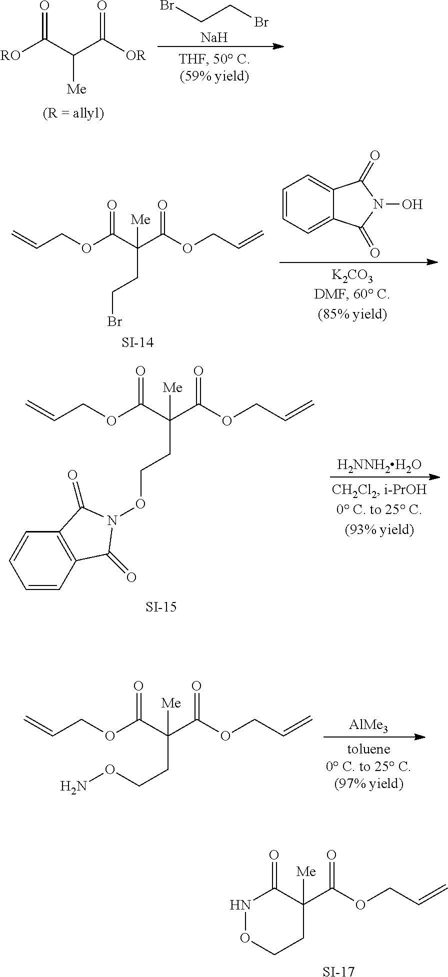

### Malonate (SI-14)

To a stirred suspension of NaH (1.23 g, 55 wt %, 28.3 mmol, 1.4 equiv) in THF (100 mL) was added diallyl 2-methylmalonate (4.00 g, 20.2 mmol, 1 equiv) at room temperature. The reaction mixture was stirred at room temperature for 20 min, and then 1,2-dibromoethane (11.4 mL, 60.5 mmol, 3.0 equiv) was added at 0° C. The reaction mixture was warmed to 50° C., stirred for 12 h and poured into a stirred mixture of 1 M HCl and diethyl ether. The phases were separated and the aqueous phase was extracted with diethyl ether. The combined organic layers were washed with saturated aqueous sodium bicarbonate and brine, dried over Na₂SO₄, filtered, and concentrated in vacuo. Flash column chromatography (SiO₂, 3% EtOAc in hexanes) malonate SI-14 (3.66 g, 12.0 mmol, 59% yield) as a colorless oil. R_{f}=0.60 (10% EtOAc in hexanes); ¹H NMR (500 MHz, CDCl₃) δ 5.88 (ddt, J=17.2, 10.4, 5.7 Hz, 2H), 5.34-5.21 (m, 4H), 4.67-4.58 (m, 4H), 3.41-3.35 (m, 2H), 2.50-2.42 (m, 2H), 1.48 (s, 3H); ¹³C NMR (126 MHz, CDCl₃) δ 170.9, 131.6, 118.9, 66.3, 54.1, 39.4, 27.1, 20.4; IR (Neat Film, NaCl) 2987, 2944, 1731, 1451, 1384, 1259, 1217, 1166, 1114, 986, 935 cm⁻¹; HRMS (ESI-APCI+) m/z calc'd for C₁₂H₁₈O₄Br [M+H]⁺: 305.0383. found 305.0382.

Malonate SI-15.

To a solution of malonate SI-14 (3.65 g, 11.9 mmol, 1 equiv) and N-hydroxyphthalimide (2.34 g, 14.4 mmol, 1.2 equiv) in DMF (50 mL) was added K₂CO₃ (2.14 g, 15.5 mmol, 1.3 equiv) at room temperature. The reaction mixture was warmed to 60° C. and stirred for 12 h. After full consumption of the starting material as indicated by TLC analysis, the reaction mixture was diluted with ethyl acetate (100 mL) and washed with 1 M HCl, saturated aqueous sodium bicarbonate and brine. The organic layers were dried over Na₂SO₄, filtered, and concentrated in vacuo. Flash column chromatography (SiO₂, 20→25% EtOAc in hexanes) afforded malonate SI-15 (3.90 g, 10.1 mmol, 85% yield) as a colorless oil. R_{f}=0.29 (25% EtOAc in hexanes); ¹H NMR (500 MHz, CDCl₃) δ 7.85-7.71 (m, 4H), 5.90-5.85 (m, 2H), 5.32-5.21 (m, 4H), 4.63-4.60 (m, 4H), 4.31 (t, J=6.9 Hz, 2H), 2.44 (t, J=6.9 Hz, 2H), 1.59 (s, 3H); ¹³C NMR (126 MHz, CDCl₃) δ 171.3, 163.5, 134.6, 131.7, 129.2, 123.7, 118.7, 75.0, 66.2, 52.5, 34.1, 20.5; IR (Neat Film, NaCl) 2948, 1790, 1732, 1467, 1374, 1240, 1188, 1124, 992, 935, 878 cm⁻¹; HRMS (ESI-APCI+) m/z calc'd for C₂₀H₂₂NO₇ [M+H]⁺: 388.1391. found 388.1387.

Alkoxyamine SI-16.

To a stirred solution of malonate SI-15 (3.87 g, 10.0 mmol, 1 equiv) in CH₂Cl₂ (45 mL) and i-PrOH (5 mL) was added hydrazine monohydrate (0.485 mL, 10.0 mmol, 1.0 equiv) at 0° C. The reaction mixture was warmed to room temperature, stirred for 15 h and filtered. The filtrate was concentrated in vacuo. Flash column chromatography (SiO₂, 20→35% EtOAc in hexanes) afforded alkoxyamine SI-16 (2.39 g, 9.29 mmol, 93% yield) as a colorless oil. R_{f}=0.19 (25% EtOAc in hexanes); ¹H NMR (500 MHz, CDCl₃) δ 5.93-5.84 (m, 2H), 5.34-5.29 (m, 2H), 5.24-5.21 (m, 2H), 4.62-4.60 (m, 4H), 3.76 (t, J=6.3 Hz, 2H), 2.22 (t, J=6.3 Hz, 2H), 1.48 (s, 3H); ¹³C NMR (126 MHz, CDCl₃) δ 171.9, 131.9, 118.5, 71.8, 66.0, 52.3, 34.3, 20.3; IR (Neat Film, NaCl) 2943, 1732, 1589, 1453, 1382, 1298, 1237, 1141, 1117, 995, 934 cm⁻¹; HRMS (ESI-APCI-O m/z calc'd for C₁₂H₂₀NO₅ [M+H]⁺: 258.1263. found 258.1333.

### 1,2-Oxazinan-3-one SI-17

To a stirred solution of alkoxyamine SI-16 (2.24 g, 8.71 mmol, 1 equiv) in toluene (87 mL) was added trimethylaluminum (8.71 mL, 2.0 M solution in toluene, 14.7 mmol, 2.0 equiv) dropwise at 0° C. The reaction mixture was warmed to room temperature, stirred for 4 h, and poured into a stirred mixture of 1 M HCl and diethyl ether. The phases were separated and the aqueous phase was extracted with ethyl acetate twice. The combined organic layers were washed with brine, dried over Na₂SO₄, filtered, and concentrated in vacuo. Flash column chromatography (SiO₂, 40→50% EtOAc in hexanes) afforded 1,2-oxazinan-3-one SI-17 (1.68 mg, 8.43 mmol, 97% yield) as a white solid. R_{f}=0.26 (33% EtOAc in hexanes); m.p. 32.6-33.3° C.; ¹H NMR (500 MHz, CDCl₃) δ 5.94-5.86 (m, 1H), 5.37-5.23 (m, 2H), 4.69-4.63 (m, 2H), 4.17 (ddd, J=10.5, 8.8, 6.7 Hz, 1H), 4.05 (ddd, J=10.5, 8.8, 4.7 Hz, 1H), 2.86 (ddd, J=13.5, 8.8, 4.7 Hz, 1H), 1.84 (ddd, J=13.5, 8.8, 6.7 Hz, 1H), 1.50 (s, 3H); ¹³C NMR (126 MHz, CDCl₃) δ 174.4, 171.5, 131.7, 118.5, 69.0, 66.3, 48.7, 33.3, 20.0; IR (Neat Film, NaCl) 3192, 2942, 1740, 1683, 1455, 1383, 1272, 1225, 1146, 979, 938 cm⁻¹; HRMS (ESI-APCI+) m/z calc'd for C₉H₁₄NO₄ [M+H]⁺: 200.0917. found 200.0920.

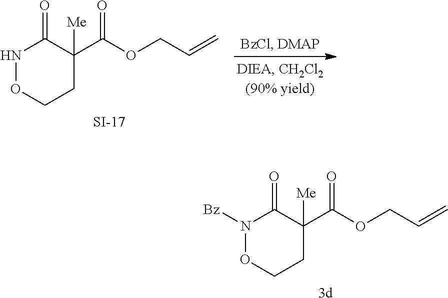

### 1,2-Oxazinan-3-one 3d

To a stirred solution of 1,2-oxazinan-3-one SI-17 (448 mg, 2.25 mmol, 1 equiv), DMAP (82.5 mg, 0.675 mmol, 0.30 equiv) and DIEA (0.980 mL, 5.63 mmol, 2.5 equiv) in CH₂Cl₂ (11.3 mL) was added benzoyl chloride (0.338 mL, 2.92 mmol, 1.3 equiv) at 0° C. The reaction mixture was warmed to room temperature, stirred for 4 h, and poured into a stirred mixture of 1 M HCl and diethyl ether. The phases were separated and the aqueous phase was extracted with ethyl acetate twice. The combined organic layers were washed with saturated aqueous sodium bicarbonate and brine. The organic layers were dried over Na₂SO₄, filtered, and concentrated in vacuo. Flash column chromatography (SiO₂, 20→30% EtOAc in hexanes) afforded 1,2-oxazinan-3-one 3d (628 mg, 2.07 mmol, 90% yield) as a colorless oil. R_{f}=0.49 (25% EtOAc in hexanes); ¹H NMR (500 MHz, CDCl₃) δ 7.69-7.64 (m, 2H), 7.55 (m, 1H), 7.45-7.41 (m, 2H), 5.94 (ddt, J=17.2, 10.4, 5.7 Hz, 1H), 5.37 (m, 1H), 5.29 (m, 1H), 4.77-4.66 (m, 2H), 4.39-4.24 (m, 2H), 3.03 (ddd, J=13.5, 9.9, 5.1 Hz, 1H), 1.90 (ddd, J=13.5, 9.4, 6.4 Hz, 1H), 1.45 (s, 3H); ¹³C NMR (126 MHz, CDCl₃) δ 170.8, 170.7, 167.0, 133.5, 132.8, 131.5, 129.4, 128.2, 119.2, 69.8, 66.7, 51.4, 32.5, 19.7; IR (Neat Film, NaCl) 2942, 1732, 1705, 1450, 1270, 1205, 1142, 981, 922, 716 cm⁻¹; HRMS (ESI-APCI+) m/z calc'd for C₁₆H₁₈NO₅ [M+H]⁺: 304.1179. found 304.1176.

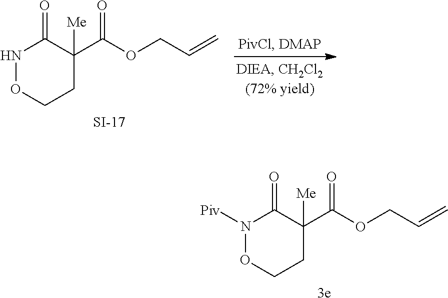

### 1,2-Oxazinan-3-one 3e

To a stirred solution of 1,2-oxazinan-3-one SI-17 (200 mg, 1.00 mmol, 1 equiv), DMAP (12.0 mg, 0.100 mmol, 0.10 equiv) and DIEA (0.435 mL, 2.51 mmol, 2.5 equiv) in CH₂Cl₂ (5.0 mL) was added pivaloyl chloride (0.213 mL, 1.31 mmol, 1.3 equiv) at 0° C. The reaction mixture was warmed to room temperature, stirred for 3 h, and poured into a stirred mixture of 1 M HCl and diethyl ether. The phases were separated and the aqueous phase was extracted with ethyl acetate twice. The combined organic layers were washed with saturated aqueous sodium bicarbonate and brine. The organic layers were dried over Na₂SO₄, filtered, and concentrated in vacuo. Flash column chromatography (SiO₂, 15→20% EtOAc in hexanes) afforded 1,2-oxazinan-3-one 3e (205 mg, 0.723 mmol, 72% yield) as a white solid. R_{f}=0.26 (25% EtOAc in hexanes); m.p. 79.8-80.2° C.; ¹H NMR (300 MHz, CDCl₃) δ 5.86 (m, 1H), 5.33-5.19 (m, 2H), 4.69-4.53 (m, 2H), 4.26 (ddd, J=10.8, 10.0, 6.6 Hz, 1H), 4.08 (ddd, J=10.8, 9.5, 4.6 Hz, 1H), 2.93 (ddd, J=13.5, 10.0, 4.6 Hz, 1H), 1.85 (ddd, J=13.5, 9.5, 6.6 Hz, 1H), 1.47 (s, 3H), 1.27 (s, 9H); ¹³C NMR (126 MHz, CDCl₃) δ 175.9, 171.0, 170.0, 131.4, 119.0, 69.3, 66.5, 50.8, 41.5, 31.9, 26.4, 19.8; IR (Neat Film, NaCl) 2980, 1763, 1734, 1273, 1235, 1195, 1137, 1116 cm⁻¹; HRMS (ESI-APCI+) m/z calc'd for C₁₄H₂₂NO₅ [M+H]⁺: 284.1492. found 284.1493.

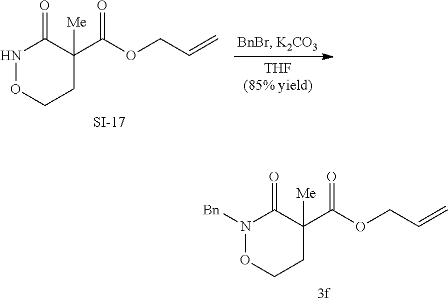

### 1,2-Oxazinan-3-one 3f

To a stirred solution of 1,2-oxazinan-3-one SI-17 (200 mg, 1.00 mmol, 1 equiv) and K₂CO₃ (350 mg, 2.51 mmol, 2.5 equiv) in THF (5.0 mL) was added benzyl bromide (0.192 mL, 2.00 mmol, 2.0 equiv) at room temperature. The reaction mixture was warmed to 50° C., stirred for 24 h, and quenched with 1 M HCl. The phases were separated, and the aqueous phase was extracted with ethyl acetate twice. The combined organic layers were washed with saturated aqueous sodium bicarbonate and brine. The organic layers were dried over Na₂SO₄, filtered, and concentrated in vacuo. Flash column chromatography (SiO₂, 15→20% EtOAc in hexanes) afforded 1,2-oxazinan-3-one 3f (246 mg, 0.850 mmol, 85% yield) as a colorless oil. R_{f}=0.30 (25% EtOAc in hexanes); ¹H NMR (300 MHz, CDCl₃) δ 7.38-7.27 (m, 5H), 5.79 (m, 1H), 5.31-5.16 (m, 2H), 4.77 (s, 2H), 4.63-4.50 (m, 2H), 4.03 (m, 1H), 3.85 (ddd, J=10.5, 9.3, 4.6 Hz, 1H), 2.81 (ddd, J=13.7, 9.3, 4.6 Hz, 1H), 1.79 (ddd, J=13.7, 9.3, 6.8 Hz, 1H), 1.49 (s, 3H); ¹³C NMR (75 MHz, CDCl₃) δ 171.7, 171.5, 135.8, 131.7, 128.6, 128.5, 127.9, 118.5, 68.4, 66.2, 49.8, 49.1, 33.6, 20.2; IR (Neat Film, NaCl) 2980, 1763, 1734, 1273, 1235, 1195, 1137, 1116 cm⁻¹; HRMS (ESI-APCI+) m/z calc'd for C₁₆H₂₀NO₄ [M+H]⁺: 290.1387. found 290.1387.

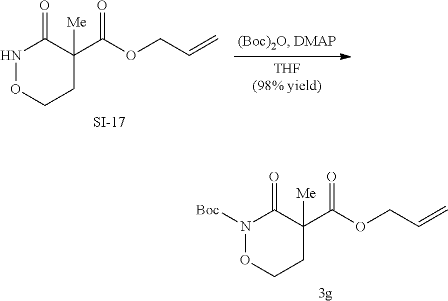

### 1,2-Oxazinan-3-one 3g

To a stirred solution of 1,2-oxazinan-3-one SI-17 (150 mg, 0.753 mmol, 1 equiv) and DMAP (18.4 mg, 0.151 mmol, 0.20 equiv) in THF (3.7 mL) was added (Boc)₂O (214 mg, 0.979 mmol, 1.3 equiv) at room temperature. After full consumption of the starting material as indicated by TLC analysis, the reaction mixture was concentrated in vacuo. Flash column chromatography (SiO₂, 15→20% EtOAc in hexanes) afforded 1,2-oxazinan-3-one 3g (220 mg, 0.735 mmol, 98% yield) as a white solid. R_{f}=0.44 (33% EtOAc in hexanes); ¹H NMR (500 MHz, CDCl₃) δ 5.87 (ddt, J=17.2, 10.5, 5.6 Hz, 1H), 5.30 (m, 1H), 5.23 (m, 1H), 4.69-4.58 (m, 2H), 4.24 (ddd, J=10.9, 9.8, 6.7 Hz, 1H), 4.12 (ddd, J=10.9, 9.5, 4.4 Hz, 1H), 2.91 (ddd, J=13.5, 9.8, 4.4 Hz, 1H), 1.84 (ddd, J=13.5, 9.5, 6.7 Hz, 1H), 1.56 (s, 9H), 1.47 (s, 3H); ¹³C NMR (126 MHz, CDCl₃) δ 170.8, 169.6, 148.2, 131.6, 118.7, 85.1, 69.1, 66.4, 51.4, 32.1, 28.2, 19.8; IR (Neat Film, NaCl) 2983, 1786, 1744, 1281, 1254, 1212, 1154, 1129 cm⁻¹; HRMS (ESI-APCI+) m/z calc'd for C₁₄H₂₁NO₆Na [M+Na]⁺: 322.1261. found 322.1248.

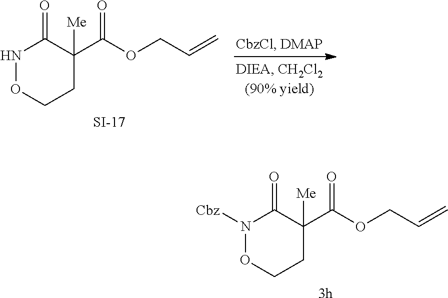

### 1,2-Oxazinan-3-one 3h

To a stirred solution of 1,2-oxazinan-3-one SI-17 (200 mg, 1.00 mmol, 1 equiv), DMAP (12.0 mg, 0.100 mmol, 0.10 equiv) and DIEA (0.435 mL, 2.51 mmol, 2.5 equiv) in CH₂Cl₂ (5.0 mL) was added benzyl chloroformate (0.184 mL, 1.31 mmol, 1.3 equiv) at 0° C. The reaction mixture was warmed to room temperature, stirred for 3 h, and poured into a stirred mixture of 1 M HCl and diethyl ether. The phases were separated and the aqueous phase was extracted with ethyl acetate twice. The combined organic layers were washed with saturated aqueous sodium bicarbonate and brine. The organic layers were dried over Na₂SO₄, filtered, and concentrated in vacuo. Flash column chromatography (SiO₂, 20→30% EtOAc in hexanes) afforded 1,2-oxazinan-3-one 3h (300 mg, 0.899 mmol, 90% yield) as a colorless oil. R_{f}=0.80 (50% EtOAc in hexanes); ¹H NMR (500 MHz, CDCl₃) δ 7.45-7.32 (m, 5H), 5.85 (ddt, J=17.3, 10.5, 5.7 Hz, 1H), 5.38-5.18 (m, 4H), 4.68-4.58 (m, 2H), 4.26 (ddd, J=10.9, 9.8, 6.8 Hz, 1H), 4.14 (ddd, J=10.9, 9.5, 4.4 Hz, 1H), 2.92 (ddd, J=13.6, 9.8, 4.4 Hz, 1H), 1.84 (ddd, J=13.6, 9.5, 6.8 Hz, 1H), 1.48 (s, 3H); ¹³C NMR (126 MHz, CDCl₃) δ 170.5, 169.4, 149.7, 134.9, 131.4, 128.8, 128.7, 128.4, 118.9, 69.4, 69.2, 66.5, 51.4, 31.9, 19.8; IR (Neat Film, NaCl) 2946, 1789, 1340, 1456, 1380, 1270, 1212, 1149, 1129, 978, 939, 753 cm⁻¹; HRMS (ESI-APCI+) m/z calc'd for C₁₇H₂₀NO₆ [M+H]⁺: 334.1285. found 334.1276.

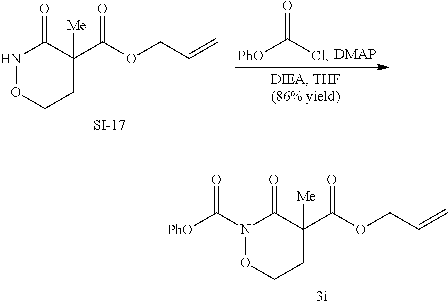

### 1,2-Oxazinan-3-one 3i

To a stirred solution of 1,2-oxazinan-3-one SI-17 (200 mg, 1.00 mmol, 1 equiv), DMAP (12.0 mg, 0.100 mmol, 0.10 equiv) and DIEA (0.435 mL, 2.51 mmol, 2.5 equiv) in CH₂Cl₂ (5.0 mL) was added phenyl chloroformate (0.164 mL, 1.31 mmol, 1.3 equiv) at 0° C. The reaction mixture was warmed to room temperature, stirred for 3 h, and poured into a stirred mixture of 1 M HCl and diethyl ether. The phases were separated and the aqueous phase was extracted with ethyl acetate twice. The combined organic layers were washed with saturated aqueous sodium bicarbonate and brine. The organic layers were dried over Na₂SO₄, filtered, and concentrated in vacuo. Flash column chromatography (SiO₂, 15→20% EtOAc in hexanes) afforded 1,2-oxazinan-3-one 3i (276 mg, 0.864 mmol, 86% yield) as a white solid. R_{f}=0.22 (25% EtOAc in hexanes); m.p. 98.0-98.3° C.; ¹H NMR (500 MHz, CDCl₃) δ 7.43-7.38 (m, 2H), 7.29-7.20 (m, 3H), 5.91 (ddt, J=17.2, 10.5, 5.7 Hz, 1H), 5.35 (m, 1H), 5.26 (m, 1H), 4.74-4.64 (m, 2H), 4.38-4.26 (m, 2H), 2.99 (ddd, J=13.7, 9.7, 4.4 Hz, 1H), 1.94 (ddd, J=13.7, 9.5, 6.9 Hz, 1H), 1.54 (s, 3H); ¹³C NMR (126 MHz, CDCl₃) δ 170.4, 169.3, 150.3, 148.0, 131.4, 129.7, 126.5, 121.4, 119.0, 69.6, 66.6, 51.5, 31.9, 19.8; IR (Neat Film, NaCl) 1797, 1757, 1739, 1294, 1268, 1218, 1187, 1163, 1145, 935, 745 cm⁻¹; HRMS (ESI-APCI+) m/z calc'd for C₁₆H₁₈NO₆ [M+H]⁺: 320.1129. found 320.1120.

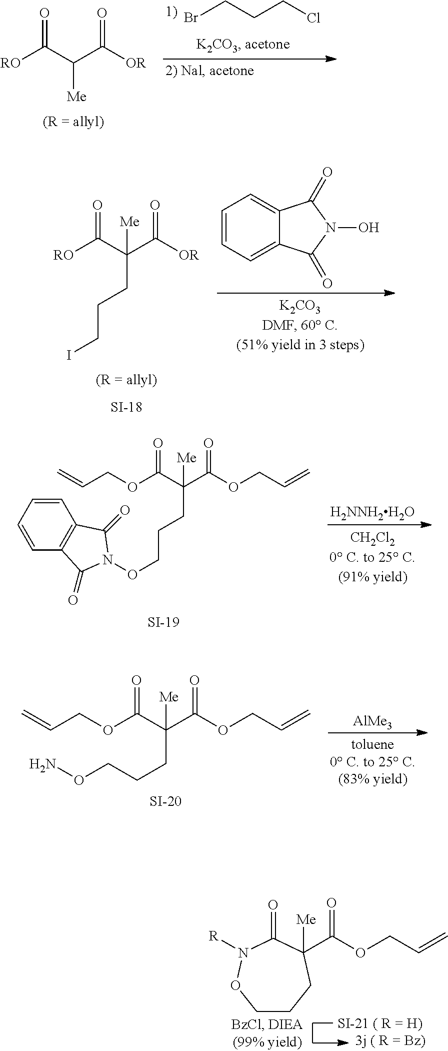

### Malonate SI-19

To a stirred suspension of K₂CO₃ (4.40 g, 31.8 mmol, 2.0 equiv) and diallyl 2-methylmalonate (3.15 g, 15.9 mmol, 1 equiv) in acetone (32 mL) was added 1-bromo-3-chloropropane (2.36 mL, 23.8 mmol, 1.5 equiv) at room temperature. The reaction mixture was stirred at 70° C. for 24 h, filtered, and concentrated in vacuo. The residue was used for the next reaction without further purification.

To a solution of the crude alkyl chloride in acetone (45 mL) was added sodium iodide (4.77 g, 31.8 mmol, 2.0 equiv) at room temperature. The reaction mixture was stirred at 70° C. for 24 h, diluted with ether, filtered, and concentrated in vacuo.

To a solution of the crude malonate SI-18 and N-hydroxyphthalimide (2.13 g, 13.1 mmol) in DMF (30 mL) was added K₂CO₃ (2.11 g, 15.3 mmol) at room temperature. The reaction mixture was warmed to 60° C. and stirred for 6 h. After full consumption of the starting material as indicated by TLC analysis, the reaction mixture was diluted with ethyl acetate (100 mL) and washed with 1 M HCl, saturated aqueous sodium bicarbonate and brine. The organic layers were dried over Na₂SO₄, filtered, and concentrated in vacuo. Flash column chromatography (SiO₂, 15→25% EtOAc in hexanes) afforded malonate SI-19 (3.27 g, 8.15 mmol, 51% yield in 3 steps) as a colorless oil. R_{f}=0.19 (20% EtOAc in hexanes); ¹H NMR (500 MHz, CDCl₃) δ 7.86-7.81 (m, 2H), 7.77-7.72 (m, 2H), 5.93-5.84 (m, 2H), 5.34-5.28 (m, 2H), 5.24-5.20 (m, 2H), 4.67-4.59 (m, 4H), 4.20 (t, J=6.5 Hz, 2H), 2.14-2.10 (m, 2H), 1.83-1.76 (m, 2H), 1.49 (s, 3H); ¹³C NMR (126 MHz, CDCl₃) δ 171.7, 163.6, 134.5, 131.8, 129.2, 123.6, 118.6, 78.4, 66.0, 53.7, 32.1, 23.7, 20.3; IR (Neat Film, NaCl) 2946, 1790, 1731, 1467, 1375, 1230, 1188, 1124, 981 cm⁻¹; HRMS (ESI-APCI+) m/z calc'd for C₂₁H₂₄NO₇ [M+H]⁺: 402.1547. found 402.1536.

Alkoxyamine SI-20.

To a stirred solution of malonate SI-19 (3.15 g, 7.85 mmol, 1 equiv) in CH₂Cl₂ (35 mL) was added hydrazine monohydrate (0.438 mL, 9.02 mmol, 1.15 equiv) at 0° C. The reaction mixture was warmed to room temperature, stirred for 7 h and filtered. The filtrate was concentrated in vacuo. Flash column chromatography (SiO₂, 20→40% EtOAc in hexanes) afforded alkoxyamine SI-20 (1.93 g, 7.11 mmol, 91% yield) as a colorless oil. R_{f}=0.38 (33% EtOAc in hexanes); ¹H NMR (500 MHz, CDCl₃) δ 5.92-5.83 (m, 2H), 5.33-5.28 (m, 2H), 5.22 (dq, J=10.5, 1.4 Hz, 2H), 4.63-4.60 (m, 4H), 3.65 (t, J=6.3 Hz, 2H), 1.96-1.91 (m, 2H), 1.59-1.52 (m, 2H), 1.45 (s, 3H); ¹³C NMR (126 MHz, CDCl₃) δ 171.8, 131.9, 118.4, 75.5, 65.8, 53.7, 32.3, 23.5, 20.1; IR (Neat Film, NaCl) 2944, 1732, 1463, 1382, 1272, 1230, 1190, 1119, 984, 935 cm⁻¹; HRMS (ESI-APCI+) m/z calc'd for C₁₃H₂₂NO₅ [M+H]⁺: 272.1492. found 272.1488.

### 1,2-Oxazepan-3-one SI-21

To a stirred solution of alkoxyamine SI-20 (1.35 g, 4.92 mmol, 1 equiv) in toluene (25 mL) was added trimethylaluminum (4.92 mL, 2.0 M solution in toluene, 9.85 mmol, 2.0 equiv) dropwise at 0° C. The reaction mixture was warmed to room temperature, stirred for 36 h, and poured into a stirred mixture of 1 M HCl and diethyl ether. The phases were separated and the aqueous phase was extracted with ethyl acetate twice. The combined organic layers were washed with brine, dried over Na₂SO₄, filtered, and concentrated in vacuo. Flash column chromatography (SiO₂, 20→30% EtOAc in hexanes) afforded 1,2-oxazepan-3-one SI-21 (874 mg, 4.10 mmol, 83% yield) as a white solid. R_{f}=0.42 (33% EtOAc in hexanes); m.p. 79.2-80.6° C.; ¹H NMR (500 MHz, CDCl₃) δ 8.18 (s, 1H), 5.89 (ddt, J=17.2, 10.6, 5.6 Hz, 1H), 5.32 (m, 1H), 5.22 (m, 1H), 4.69-4.61 (m, 2H), 4.16 (m, 1H), 3.82 (ddd, J=11.9, 10.3, 3.4 Hz, 1H), 2.34-2.22 (m, 2H), 1.82-1.64 (m, 2H), 1.46 (s, 3H); ¹³C NMR (126 MHz, CDCl₃) δ 176.4, 172.0, 132.0, 118.3, 76.4, 65.8, 51.9, 31.8, 25.5, 24.1; IR (Neat Film, NaCl) 3184, 3065, 1733, 1662, 1451, 1258, 1217, 1140, 1084, 970, 920 cm⁻¹; HRMS (ESI-APCI+) m/z calc'd for C₁₀H₁₆NO₄ [M+H]⁺: 214.1074. found 214.1070.

### 1,2-Oxazepan-3-one 3j

To a stirred solution of 1,2-oxazepan-3-one SI-21 (300 mg, 1.41 mmol, 1 equiv), DMAP (17.0 mg, 0.141 mmol, 0.10 equiv) and DIEA (0.614 mL, 3.53 mmol, 2.5 equiv) in CH₂Cl₂ (7.0 mL) was added benzoyl chloride (0.197 mL, 1.69 mmol, 1.2 equiv) at 0° C. The reaction mixture was stirred at 0° C. for 1 h, poured into a stirred mixture of 1 M HCl and diethyl ether. The phases were separated and the aqueous phase was extracted with ethyl acetate twice. The combined organic layers were washed with saturated aqueous sodium bicarbonate and brine. The organic layers were dried over Na₂SO₄, filtered, and concentrated in vacuo. Flash column chromatography (SiO₂, 20% EtOAc in hexanes) afforded 1,2-oxazepan-3-one 3j (443 mg, 1.40 mmol, 99% yield) as a colorless oil. R_{f}=0.50 (33% EtOAc in hexanes); ¹H NMR (500 MHz, CDCl₃) δ 7.65-7.61 (m, 2H), 7.53 (m, 1H), 7.44-7.40 (m, 2H), 5.96 (ddt, J=17.4, 10.4, 5.9 Hz, 1H), 5.38 (m, 1H), 5.28 (m, 1H), 4.76-4.67 (m, 2H), 4.31 (ddd, J=12.0, 7.3, 4.8 Hz, 1H), 4.20 (ddd, J=12.0, 7.1, 4.3 Hz, 1H), 2.50 (ddd, J=14.5, 8.5, 4.8 Hz, 1H), 2.20 (m, 1H), 1.85 (m, 1H), 1.62 (ddd, J=14.5, 7.8, 4.6 Hz, 1H), 1.44 (s, 3H); ¹³C NMR (126 MHz, CDCl₃) δ 172.5, 171.6, 168.3, 134.2, 132.4, 131.9, 128.7, 128.2, 119.1, 76.4, 66.3, 53.7, 31.2, 24.5, 23.9; IR (Neat Film, NaCl) 2940, 1722, 1704, 1449, 1261, 1226, 1212, 1138, 993, 928 cm⁻¹; HRMS (ESI-APCI+) m/z calc'd for C₁₇H₂₀NO₅ [M+H]⁺: 318.1336. found 318.1339.

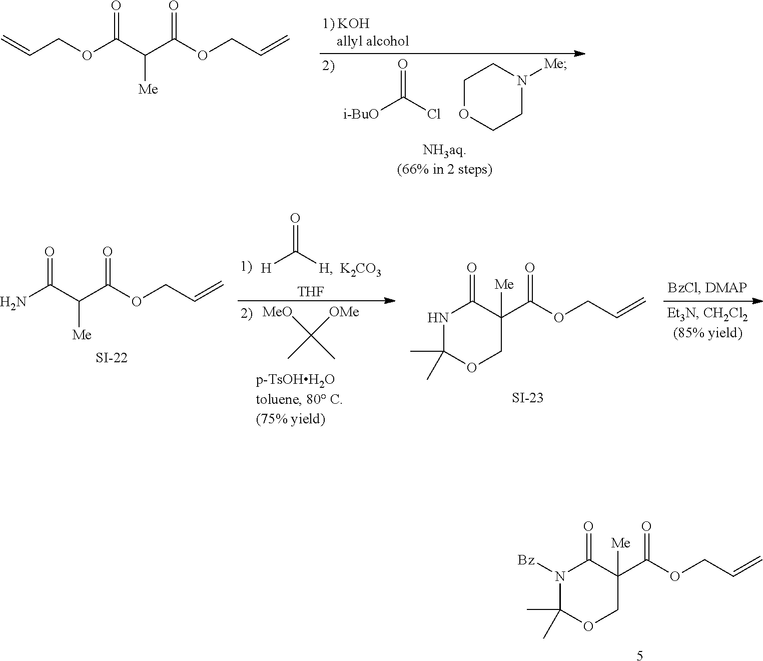

### Amide SI-22

To a solution of diallyl 2-methylmalonate (2.00 g, 10.1 mmol, 1 equiv) in allyl alcohol (10 mL) was added a solution of KOH (623 mg, 11.1 mmol, 1.1 equiv) in allyl alcohol (10 mL) at room temperature. After full consumption of the starting material as indicated by TLC analysis, the reaction mixture was quenched with 1 M HCl and the aqueous phase was extracted with CH₂Cl₂ three times. The combined organic layers were dried over Na₂SO₄, filtered, and concentrated in vacuo. The residue was used for the next reaction without further purification. To a solution of the crude acid in diethyl ether (30 mL) was added N-methylmorpholine (1.17 mL, 10.6 mmol, 1.05 equiv) and isobutyl chloroformate (1.45 mL, 10.6 mmol, 1.05 equiv) at 0° C. After stirring at 0° C. for 10 min, the reaction mixture was filtered and the filtrate was poured into a stirred solution of aqueous ammonia (2.2 mL, 26 wt %, 30.3 mmol, 3.0 equiv) in THF (10 mL) at 0° C. After 10 min, the reaction mixture was diluted with CH₂Cl₂ and the phases were separated. The aqueous phase was extracted with CH₂Cl₂ three times. The combined organic layers were washed with brine, dried over Na₂SO₄, filtered, and concentrated in vacuo. Flash column chromatography (SiO₂, 40→60% EtOAc in hexanes) afforded amide SI-22 (1.05 g, 6.68 mmol, 60% yield in 2 steps) as a white solid. R_{f}=0.23 (50% EtOAc in hexanes); m.p. 53.9-54.3° C.; ¹H NMR (500 MHz, CDCl₃) δ 5.91 (ddt, J=17.3, 10.5, 5.8 Hz, 1H), 5.38-5.22 (m, 2H), 4.67-4.60 (m, 2H), 3.36 (q, J=7.3 Hz, 1H), 1.47 (d, J=7.3 Hz, 3H); ¹³C NMR (126 MHz, CDCl₃) δ 171.9, 171.3, 131.5, 119.1, 66.3, 46.7, 15.1; IR (Neat Film, NaCl) 3425, 3332, 3198, 1735, 1672, 1615, 1456, 1397, 1260, 1185, 1096, 932 cm⁻¹; HRMS (ESI-APCI+) m/z calc'd for C₇H₁₂NO₃ [M+H]⁺: 158.0812. found 158.0814.

### 1,3-Oxazinan-4-one SI-23

To a solution of amide SI-22 (455 mg, 2.89 mmol, 1 equiv) in THF (6 mL) was added K₂CO₃ (79.9 mg, 0.578 mmol, 0.20 equiv) and formaldehyde (0.352 mL, 37% aqueous solution, 4.34 mmol, 1.5 equiv) at room temperature. After full consumption of the starting material as indicated by TLC analysis, the reaction mixture was diluted with ethyl acetate (50 mL), dried over Na₂SO₄, filtered, and concentrated in vacuo. The residue was used for the next reaction without further purification.

To a solution of the crude alcohol in toluene (9 mL) added 2,2-dimethoxypropane (3.5 mL, 28.9 mmol, 10 equiv) and p-toluenesulfonic acid monohydrate (27.6 mg, 0.145 mmol, 0.05 equiv) at room temperature. After stirring at 80° C. for 12 h, the reaction mixture was diluted with ethyl acetate (30 mL), washed with saturated aqueous sodium bicarbonate and brine, dried over Na₂SO₄, filtered, and concentrated in vacuo. Flash column chromatography (SiO₂, 50% EtOAc in hexanes) afforded 1,3-oxazinan-4-one SI-23 (494 mg, 2.17 mmol, 75% yield in 2 steps) as a white solid. R_{f}⁼0.54 (66% EtOAc in hexanes); m.p. 30.6-31.2° C.; ¹H NMR (500 MHz, CDCl₃) δ 6.90 (s, 1H), 5.90 (ddt, J=17.3, 10.5, 5.6 Hz, 1H), 5.38-5.18 (m, 2H), 4.73-4.59 (m, 2H), 4.23 (d, J=11.7 Hz, 1H), 3.76 (d, J=11.7 Hz, 1H), 1.48 (s, 3H), 1.45 (s, 3H), 1.44 (s, 3H); ¹³C NMR (126 MHz, CDCl₃) δ 171.4, 169.0, 131.7, 118.6, 86.2, 67.2, 66.3, 50.9, 29.1, 27.7, 17.7; IR (Neat Film, NaCl) 3198, 1735, 1672, 1412, 1370, 1245, 1201, 1127, 1082 cm⁻¹; HRMS (ESI-APCI+) m/z calc'd for C₁₁H₁₈NO₄ [M+H]⁺: 228.1230. found 228.1232.

### 1,3-Oxazinan-4-one 5

To a stirred solution of 1,3-oxazinan-4-one SI-23 (366 mg, 1.61 mmol, 1 equiv), DMAP (19.7 mg, 0.161 mmol, 0.10 equiv) and Et₃N (0.561 mL, 4.03 mmol, 2.5 equiv) in CH₂Cl₂ (8.0 mL) was added benzoyl chloride (0.224 mL, 1.61 mmol, 1.2 equiv) at 0° C. The reaction mixture was warmed to room temperature, stirred for 48 h, and diluted with diethyl ether (50 mL). The organic layers were washed with 1 M HCl, saturated aqueous sodium bicarbonate and brine, dried over Na₂SO₄, filtered, and concentrated in vacuo. Flash column chromatography (SiO₂, 10% EtOAc in hexanes) afforded 1,3-oxazinan-4-one 5 (455 mg, 1.37 mmol, 85% yield) as a white solid. R_{f}=0.40 (25% EtOAc in hexanes); m.p. 67.0-67.4° C.; ¹H NMR (500 MHz, CDCl₃) δ 7.88-7.82 (m, 2H), 7.53 (m, 1H), 7.45-7.37 (m, 2H), 5.98 (ddt, J=17.2, 10.4, 5.8 Hz, 1H), 5.44-5.28 (m, 2H), 4.81-4.67 (m, 2H), 4.40 (d, J=12.1 Hz, 1H), 3.96 (d, J=12.1 Hz, 1H), 1.84 (s, 3H), 1.69 (s, 3H), 1.47 (s, 3H); ¹³C NMR (126 MHz, CDCl₃) δ 175.2, 171.1, 170.3, 136.0, 132.8, 131.5, 128.6, 128.6, 119.4, 93.1, 66.8, 66.7, 52.2, 27.3, 26.8, 18.1; IR (Neat Film, NaCl) 2988, 2940, 1738, 1701, 1685, 1450, 1389, 1322, 1263, 1247, 1163, 1141, 1084, 978, 816, 718 cm⁻¹; HRMS (ESI-APCI+) m/z calc'd for C₁₈H₂₂NO₅ [M+H]⁺: 332.1492. found 332.1485.

### Example 2

### Representative Procedure for Palladium-Catalyzed Allylic Alkylation

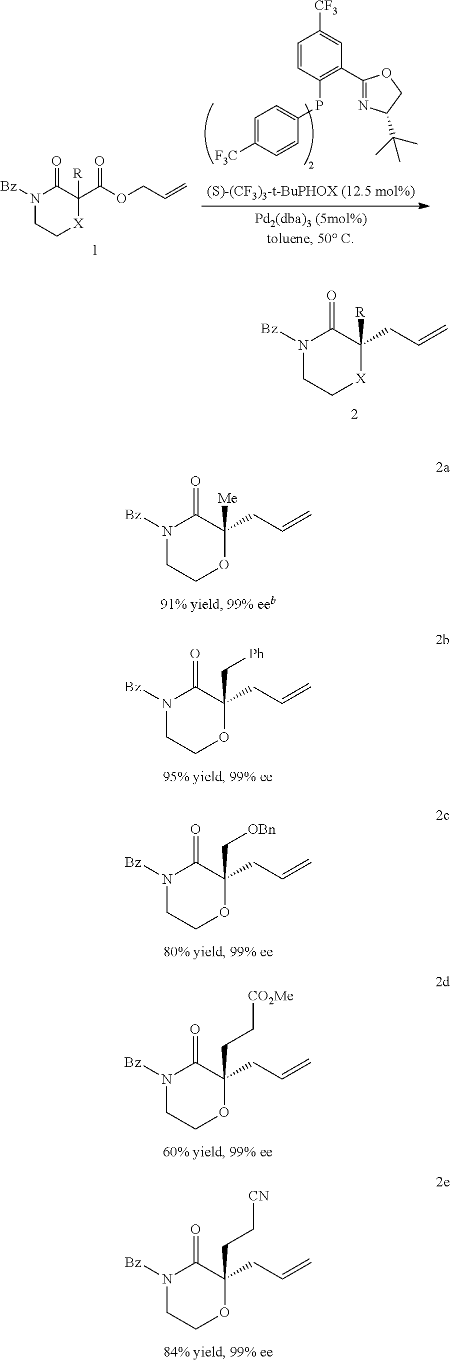

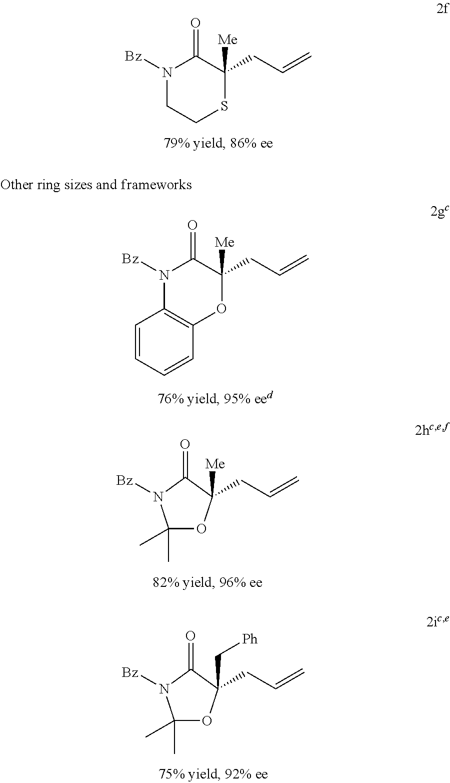

The racemic morpholinone substrates of Example 1 were subjected to palladium-catalyzed decarboxylative allylic alkylation with Pd₂(dba)₃ (5 mol %) and (S)—(CF₃)₃-t-BuPHOX ligand (12.5 mol %, PHOX=phosphinooxazoline) in a 0.033 M solution of toluene. Simple α-benzyl substitution performed well in this chemistry; the desired 2-benzyl α-tetrasubstituted morpholinone 2b was obtained in 95% yield and 99% ee. Gratifyingly, other functionalized substrates (benzylether, methyl ester, nitrile) are well tolerated, affording α-functionalized morpholinones 2c, 2d and 2e in uniformly excellent enantioenrichment (99% ee), although the yield of 2d was moderate (60%). Having demonstrated a broad functional group tolerance within the side chain, other ring sizes and frameworks were explored. Replacement of oxygen with sulfur gave thiomorpholinone 2f in good yield, but slightly decreased enantioselectivity (79% yield, 86% ee). Like morpholinone, benzomorpholinone is also a good substrate class, delivering allylated product 2g in 76% yield and 95% ee. Additionally, α-tetrasubstituted oxazolidin-4-one 2h is produced in 82% yield and 96% ee with higher temperature applied (60° C.). Benzyloxazolinone 2i is also produced in good yield and enantioselectivity.

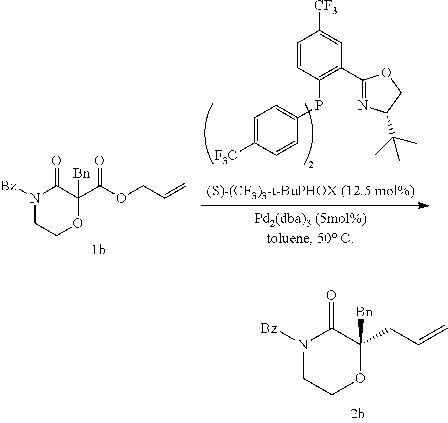

In a nitrogen-filled glove box, Pd₂(dba)₃ (4.6 mg, 0.005 mmol, 5 mol %) and (S)—(CF₃)₃-t-BuPHOX (7.4 mg, 0.0125 mmol, 12.5 mol %) were added to a 20 mL scintillation vial equipped with a magnetic stirring bar. The vial was then charged with toluene (2.0 mL) and stirred at 25° C. for 30 min, generating a yellow solution. To the above preformed catalyst solution was added a solution of 1b (37.9 mg, 0.10 mmol, 1 equiv) in toluene (1.0 mL). The vial was sealed and stirred at 50° C. until 1b was fully consumed by TLC analysis. The reaction mixture was concentrated in vacuo. Flash column chromatography (SiO₂, 10% EtOAc in hexanes) afforded morpholinone 2b (31.8 mg, 94.8 mmol, 95% yield) as a colorless oil. 99% ee, [α]_{D}²⁵+85.9 (c 1.15, CHCl₃); R_{f}=0.63 (25% EtOAc in hexanes); ¹H NMR (500 MHz, CDCl₃) δ 7.50 (m, 1H), 7.42-7.34 (m, 3H), 7.34-7.23 (m, 6H), 5.94 (ddt, J=16.9, 10.3, 7.2 Hz, 1H), 5.25-5.16 (m, 2H), 4.01 (ddd, J=12.2, 7.0, 3.1 Hz, 1H), 3.85 (ddd, J=12.2, 6.3, 3.2 Hz, 1H), 3.77 (ddd, J=13.0, 6.3, 3.1 Hz, 1H), 3.50 (ddd, J=13.0, 7.0, 3.2 Hz, 1H), 3.21 (d, J=13.7 Hz, 1H), 2.93 (d, J=13.7 Hz, 1H), 2.75 (m, 1H), 2.45 (m, 1H); ¹³C NMR (126 MHz, CDCl₃) δ 173.0, 172.7, 136.1, 135.9, 132.3, 131.8, 131.0, 128.3, 128.1, 128.0, 127.2, 119.7, 83.7, 60.8, 45.1, 43.7, 43.2; IR (Neat Film, NaCl) 3062, 3029, 2976, 2927, 1686, 1462, 1448 1369, 1300, 1282, 1220, 1091, 1023, 923, 726, 700 cm⁻¹; HRMS (ESI-APCI+) m/z calc'd for C₂₁H₂₂NO₃ [M+H]⁺: 336.1594. found 336.1594; SFC conditions: 10% MeOH, 3.0 mL/min, Chiralpak AD-H column, λ=254 nm, t_{R} (min): major=3.67, minor=5.93.

### Spectroscopic Data for Exemplary Alkylation Products

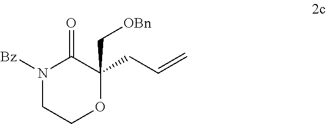

### Morpholinone 2c

Flash column chromatography (SiO₂, 15% EtOAc in hexanes) afforded morpholinone 2c (80% yield) as a colorless oil. 99% ee, [α]_{D}²⁵ −22.4 (c 1.37, CHCl₃); R_{f}=0.42 (33% EtOAc in hexanes); ¹H NMR (500 MHz, CDCl₃) δ 7.64-7.57 (m, 2H), 7.46 (m, 1H), 7.40-7.26 (m, 7H), 5.85 (ddt, J=16.8, 10.4, 7.2 Hz, 1H), 5.19-5.10 (m, 2H), 4.62 (d, J=12.0 Hz, 1H), 4.57 (d, J=12.0 Hz, 1H), 4.44 (ddd, J=12.1, 7.2, 4.0 Hz, 1H), 4.07 (ddd, J=12.1, 5.1, 3.6 Hz, 1H), 4.00-3.90 (m, 2H), 3.89 (d, J=9.5 Hz, 1H), 3.63 (d, J=9.5 Hz, 1H), 2.58 (m, 1H), 2.38 (m, 1H); ¹³C NMR (126 MHz, CDCl₃) δ 173.3, 172.1, 137.8, 135.6, 131.9, 131.7, 128.6, 128.4, 128.1, 127.9, 127.7, 119.7, 82.9, 77.6, 74.1, 62.0, 45.6, 40.1; IR (Neat Film, NaCl) 3062, 3029, 3894, 3863, 1686, 1462, 1449, 1371, 1325, 1283, 1226, 1116, 1088, 923, 728, 696 cm⁻¹; HRMS (ESI+) m/z calc'd for C₂₂H₂₄NO₄ [M+H]⁺: 366.1700. found 366.1703; SFC conditions: 5% MeOH, 3.0 mL/min, Chiralpak AD-H column, λ=254 nm, t_{R} (min): major=5.91, minor=6.73.

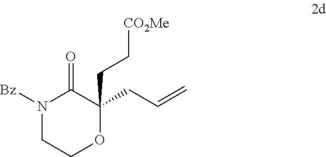

### Morpholinone 2d

Flash column chromatography (SiO₂, 20→25% EtOAc in hexanes) afforded morpholinone 2d (60% yield) as a colorless oil. 99% ee, [α]_{D}²⁵+24.6 (c 1.15, CHCl₃); R_{f}=0.25 (25% EtOAc in hexanes); ¹H NMR (500 MHz, CDCl₃) δ 7.59-7.46 (m, 3H), 7.44-7.36 (m, 2H), 5.86 (ddt, J=16.9, 10.4, 7.2 Hz, 1H), 5.24-5.14 (m, 2H), 4.08 (ddd, J=12.4, 7.0, 3.6 Hz, 1H), 4.02 (ddd, J=12.4, 6.0, 3.5 Hz, 1H), 3.95 (ddd, J=13.0, 7.0, 3.4 Hz, 1H), 3.89 (ddd, J=13.0, 6.0, 3.6 Hz, 1H), 3.67 (s, 3H), 2.69 (m, 1H), 2.56-2.41 (m, 2H), 2.37 (ddd, J=15.7, 9.4, 5.9 Hz, 1H), 2.29 (ddd, J=14.3, 9.5, 5.9 Hz, 1H), 2.15-2.02 (m, 1H); ¹³C NMR (126 MHz, CDCl₃) δ 173.6, 173.3, 173.0, 135.8, 132.0, 131.8, 128.3, 127.9, 119.8, 81.8, 59.8, 51.9, 45.6, 41.0, 31.7, 28.6; IR (Neat Film, NaCl) 2951, 1737, 1687, 1438, 1369, 1283, 1226, 1124, 924 cm⁻¹; HRMS (ESI-APCI+) m/z calc'd for C₁₈H₂₂NO₅ [M+H]⁺: 332.1492. found 332.1494; SFC conditions: 5% MeOH, 3.0 mL/min, Chiralcel OD-H column, λ=254 nm, t_{R} (min): major=6.16, minor=5.66.

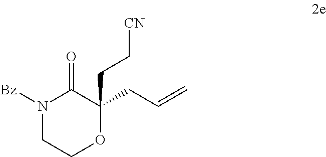

### Morpholinone 2e

Flash column chromatography (SiO₂, 20→25% EtOAc in hexanes) afforded morpholinone 2e (84% yield) as a colorless oil. 99% ee, [α]_{D}²⁵+38.5 (c 1.24, CHCl₃); R_{f}=0.23 (25% EtOAc in hexanes); ¹H NMR (500 MHz, CDCl₃) δ 7.63-7.48 (m, 3H), 7.48-7.35 (m, 2H), 5.84 (ddt, J=17.3, 10.2, 7.2 Hz, 1H), 5.29-5.18 (m, 2H), 4.18 (ddd, J=12.9, 8.2, 3.3 Hz, 1H), 4.08-3.84 (m, 3H), 2.76 (m, 1H), 2.58-2.43 (m, 2H), 2.38 (ddd, J=16.7, 9.3, 6.2 Hz, 1H), 2.28 (ddd, J=14.2, 9.3, 6.2 Hz, 1H), 2.07 (ddd, J=14.2, 9.3, 5.9 Hz, 1H); ¹³C NMR (126 MHz, CDCl₃) δ 173.1, 172.3, 135.5, 132.2, 131.0, 128.4, 128.0, 120.5, 119.3, 80.9, 59.8, 45.6, 40.8, 32.1, 12.1; IR (Neat Film, NaCl) 3075, 2928, 2247, 1687, 1370, 1283, 1229, 1128, 1091, 926, 727, 696 cm⁻¹; HRMS (ESI-APCI+) m/z calc'd for C₁₇H₁₉N₂O₃ [M+H]⁺: 299.1390. found 299.1383; SFC conditions: 3% MeOH, 2.5 mL/min, Chiralpak AS-H column, λ=254 nm, t_{R} (min): major=5.79, minor=6.53.

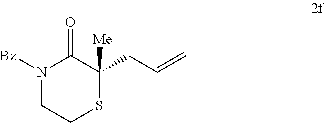

### Thiomorpholinone 2f

Flash column chromatography (SiO₂, 12→15% EtOAc in hexanes) afforded thiomorpholinone 2f (79% yield) as a colorless oil. 86% ee, [α]_{D}²⁵ −45.8 (c 1.35, CHCl₃); R_{f}=0.48 (25% EtOAc in hexanes); ¹H NMR (500 MHz, CDCl₃) δ 7.58-7.45 (m, 3H), 7.45-7.36 (m, 2H), 5.86 (dddd, J=16.6, 10.4, 7.5, 6.8 Hz, 1H), 5.23-5.14 (m, 2H), 4.30-4.16 (m, 2H), 3.11-2.98 (m, 2H), 2.81 (m, 1H), 2.58 (m, 1H), 1.58 (s, 3H); ¹³C NMR (126 MHz, CDCl₃) δ 175.7, 175.5, 136.1, 132.3, 131.8, 128.5, 127.5, 119.7, 50.4, 48.9, 43.5, 25.6, 23.9; IR (Neat Film, NaCl) 3075, 2977, 2931, 2359, 1683, 1448, 1382, 1305, 1280, 1214, 1139, 986, 922, 725, 693, 666 cm⁻¹; HRMS (ESI-APCI+) m/z calc'd for C₁₅H₁₈NO₂S [M+H]⁺: 276.1053. found 276.1051; SFC conditions: 5% MeOH, 3.0 mL/min, Chiralcel OJ-H column, λ=254 nm, t_{R} (min): major=6.80, minor=5.74.

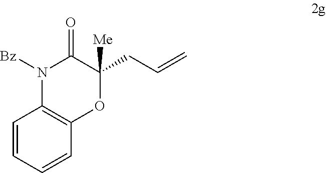

### Benzomorpholinone 2g

Flash column chromatography (SiO₂, 5% EtOAc in hexanes) afforded benzomorpholinone 2g (76% yield) as a colorless oil. [α]_{D}²⁵ −10.4 (c 0.27, CHCl₃); R_{f}=0.31 (10% EtOAc in hexanes); ¹H NMR (500 MHz, CDCl₃) δ 7.90-7.87 (m, 2H), 7.62 (m, 1H), 7.49-7.45 (m, 2H), 7.07 (m, 1H), 7.06 (d, J=1.1 Hz, 1H), 6.96-6.88 (m, 2H), 5.89 (m, 1H), 5.22-5.14 (m, 2H), 2.76 (ddt, J=14.3, 6.8, 1.3 Hz, 1H), 2.55 (ddt, J=14.3, 7.6, 1.2 Hz, 1H), 1.56 (s, 3H); ¹³C NMR (126 MHz, CDCl₃) δ 171.8, 168.6, 143.0, 134.6, 133.4, 131.3, 130.3, 129.2, 127.4, 125.5, 122.8, 120.0, 118.5, 116.8, 80.3, 40.4, 21.5; IR (Neat Film, NaCl) 1723, 1695, 1498, 1353, 1282, 1258, 750 cm⁻¹; HRMS (ESI-APCI+) m/z calc'd for C₁₉H₁₈NO₃ [M+H]⁺: 308.1281. found 308.1275.

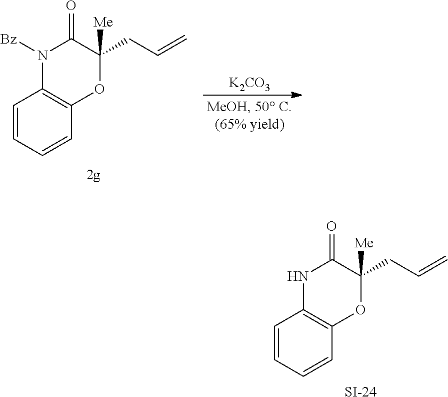

### Benzomorpholinone SI-24

To a solution of 2g (13.1 mg, 42.6 μmol, 1 equiv) in MeOH (2 mL) was added K₂CO₃ (7.1 mg, 51 μmol, 1.2 equiv) at room temperature. After stirring at 50° C. for 8 h, the reaction mixture was filtered and the filtrate was concentrated in vacuo. Flash column chromatography (SiO₂, 20% EtOAc in hexanes) afforded benzomorpholinone SI-24 (5.6 mg, 27.6 μmol, 65% yield) as a white solid. 95% ee, [α]_{D}²⁵ −15.2 (c 0.21, CHCl₃); R_{f}=0.36 (20% EtOAc in hexanes); ¹H NMR (500 MHz, CDCl₃) δ 8.13 (s, 1H), 7.00-6.91 (m, 3H), 6.76 (m, 1H), 5.87 (dddd, J=16.5, 10.8, 7.6, 6.7 Hz, 1H), 5.16-5.11 (m, 2H), 2.69 (m, 1H), 2.49 (ddt, J=14.3, 7.7, 1.2 Hz, 1H), 1.52 (s, 3H); ¹³C NMR (126 MHz, CDCl₃) δ 169.3, 142.6, 131.7, 126.7, 124.3, 122.4, 119.5, 117.7, 115.1, 80.5, 41.0, 21.8; IR (Neat Film, NaCl) 3206, 3077, 2982, 2919, 1687, 1611, 1502, 1379, 1279, 750 cm⁻¹; HRMS (ESI-APCI+) m/z calc'd for C₁₂H₁₄NO₂ [M+H]⁺: 204.1019. found 204.1016; SFC conditions: 5% IPA, 3.0 mL/min, Chiralcel OJ-H column, λ=210 nm, t_{R} (min): major=4.18, minor=4.64.

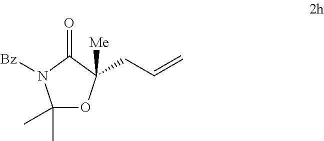

### Oxazolidinone 2h

Flash column chromatography (SiO₂, 7→10% EtOAc in hexanes) afforded oxazolidinone 2h (82% yield) as a colorless oil. 96% ee, [α]_{D}²⁵+68.2 (c 1.05, CHCl₃); R_{f}=0.47 (15% EtOAc in hexanes); ¹H NMR (500 MHz, CDCl₃) δ 7.58-7.52 (m, 3H), 7.41 (ddt, J=7.8, 6.6, 1.1 Hz, 2H), 5.94 (m, 1H), 5.29-5.20 (m, 2H), 2.54-2.44 (m, 2H), 1.83 (s, 3H), 1.76 (s, 3H), 1.46 (s, 3H); ¹³C NMR (126 MHz, CDCl₃) δ 174.4, 169.6, 134.7, 132.4, 132.4, 128.8, 128.1, 119.8, 95.1, 81.1, 43.7, 29.2, 27.7, 25.1; IR (Neat Film, NaCl) 2985, 1753, 1689, 1371, 1336, 1304, 1284, 1210, 1181, 997 cm⁻¹; HRMS (ESI-APCI+) m/z calc'd for C₁₆H₂₀NO₃ [M+H]⁺: 274.1438. found 274.1434; SFC conditions: 2% IPA, 3.0 mL/min, Chiralcel OJ-H column, λ=254 nm, t_{R} (min): major=5.59, minor=3.96.

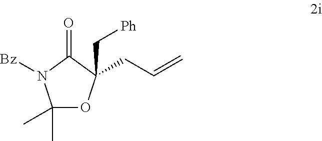

### Oxazolidinone 2i

Flash column chromatography (SiO₂, 5→7% EtOAc in hexanes) afforded oxazolidinone 2i (75% yield) as a colorless oil. 92% ee, [α]_{D}²⁵+46.9 (c 1.03, CHCl₃); R_{f}=0.35 (10% EtOAc in hexanes); ¹H NMR (500 MHz, CDCl₃) δ 7.52 (m, 1H), 7.40-7.29 (m, 7H), 7.24-7.20 (m, 2H), 5.98 (m, 1H), 5.30-5.22 (m, 2H), 3.04 (d, J=14.4 Hz, 1H), 3.01 (d, J=14.4 Hz, 1H), 2.63-2.50 (m, 2H), 1.82 (s, 3H), 1.45 (s, 3H); ¹³C NMR (126 MHz, CDCl₃) δ 173.2, 169.6, 135.7, 134.7, 132.3, 132.0, 131.5, 128.7, 128.2, 128.0, 127.3, 120.0, 95.5, 84.4, 43.0, 42.9, 29.1, 27.6; IR (Neat Film, NaCl) 1750, 1688, 1346, 1303, 1282 1125, 922 cm⁻¹; HRMS (ESI-APCI+) m/z calc'd for C₂₂H₂₄NO₃ [M+H]⁺: 350.1751. found 350.1751; SFC conditions: 5% MeOH, 3.0 mL/min, Chiralcel OJ-H column, λ=254 nm, t_{R} (min): major=3.51, minor=4.44.

With excellent results on α,α-dialkyl 2-oxa- and thia-linked lactams in hand, the allylic alkylation using cyclic hydroxamic acid derivatives to obtain α-quaternary N,O-heterocycles was investigated (Table 1). Isoxazolidin-3-one 3a (R=Bz), 3b (R=Boc) and 3c (R=PhO(CO)) produced the desired alkylated compounds 4a-c in excellent yields (95-98%), but with modest enantioselectivities (72-73% ee) (entries 1-3). Benzoyl protected 1,2-oxazinan-3-one 3d underwent an unexpected side reaction, and produced only small amounts of 4d (entry 4). Despite of the low yield, the enantioselectivity of 4d is still satisfactory (88% ee), which encouraged us to identify an effective N-protecting group to circumvent the undesired reaction. A bulky pivaloyl group somehow suppresses the side reaction, but decreases the enantioselectivity (entry 5). An electron-rich N-benzylated 3f was a poor substrate for decarboxylative alkylation (entry 6). Finally, carbamates 3g-i produced the desired products in good yields (67-89%) and acceptable enantioselectivities (84-87% ee) (entries 7-9), with little or none of the undesired side reactivity observed. 7-membered ring substrate 3j is an excellent substrate in this class, furnishing 4j in a good yield and excellent enantioselectivity (entry 10, 81% yield, 93% ee).

TABLE 1
Substrate Scope of α-Quaternary Cyclic Hydroxamic Acid Derivatives

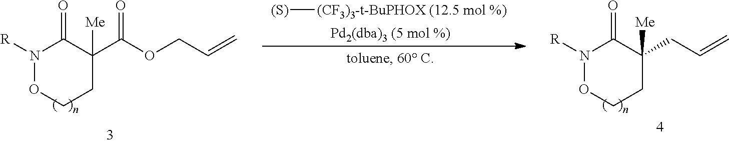

| entry | substrate | R | yield (%)^{b} | ee (%)^{c} |
|---|---|---|---|---|
| 1 2 3 | 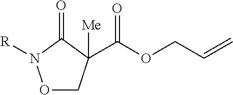 | Bz (3a→4a) Boc (3b→4b) PhO(CO) (3c→4c) | 98 95 95 | 73 72 73 |
| 4 5 6 7 8 9 | 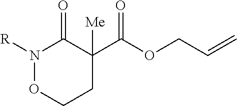 | Bz (3d→4d) Piv (3e→4e) Bn (3f→4f) Boc (3g→4g) Cbz (3h→4h) PhO(CO) (3i→4i) | 29 48 trace 67 89 70 | 88 73 ND 85 84 87 |
| 10 | 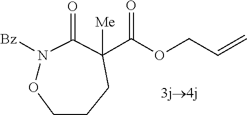 | | 81 | 93 |

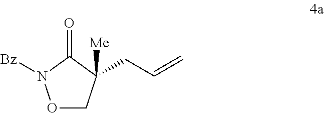

### Isoxazolidinone 4a

Flash column chromatography (SiO₂, 15→20% EtOAc in hexanes) afforded isoxazolidinone 4a (95% yield) as a colorless oil. 73% ee, [α]_{D}²⁵ −33.5 (c 1.05, CHCl₃); R_{f}=0.34 (25% EtOAc in hexanes); ¹H NMR (500 MHz, CDCl₃) δ 7.73-7.70 (m, 2H), 7.57 (m, 1H), 7.47-7.42 (m, 2H), 5.79 (ddt, J=16.8, 10.2, 7.4 Hz, 1H), 5.24-5.17 (m, 2H), 4.40 (d, J=8.7 Hz, 1H), 4.16 (d, J=8.7 Hz, 1H), 2.48 (m, 1H), 2.38 (m, 1H), 1.33 (s, 3H); ¹³C NMR (126 MHz, CDCl₃) δ 173.0, 164.0, 133.0, 132.2, 131.7, 129.8, 128.1, 120.3, 76.7, 47.5, 39.4, 19.7; IR (Neat Film, NaCl) 1758, 1696, 1449, 1276, 1230, 1144, 993 cm⁻¹; HRMS (ESI-APCI+) m/z calc'd for C₁₄H₁₆NO₃ [M+H]⁺: 246.1125. found 246.1116; SFC conditions: 5% MeOH, 3.0 mL/min, Chiralcel OJ-H column, λ=254 nm, t_{R} (min): major=8.07, minor=5.70.

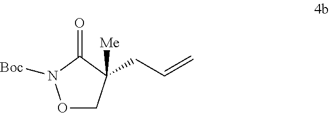 [SMILES: C=CC[C@]1(C)CON(C(=O)OC(C)(C)C)C1=O]

### Isoxazolidinone 4b

Flash column chromatography (SiO₂, 10→15% EtOAc in hexanes) afforded isoxazolidinone 4b (98% yield) as a colorless oil. 72% ee, [α]_{D}²⁵ −27.7 (c 1.10, CHCl₃); R_{f}=0.38 (25% EtOAc in hexanes); ¹H NMR (500 MHz, CDCl₃) δ 5.75 (m, 1H), 5.20-5.14 (m, 2H), 4.25 (d, J=8.6 Hz, 1H), 4.01 (d, J=8.6 Hz, 1H), 2.45-2.33 (m, 2H), 1.57 (s, 9H), 1.27 (s, 3H); ¹³C NMR (126 MHz, CDCl₃) δ 171.6, 146.6, 131.9, 120.0, 85.2, 76.7, 47.2, 39.2, 28.2, 19.5; IR (Neat Film, NaCl) 2981, 1785, 1747, 1370, 1305, 1256, 1157, 1106, 990 cm⁻¹; HRMS (ESI-APCI+) m/z calc'd for C₁₂H₂₃N₂O₄ [M+NH₄]′: 259.1652. found 259.1641; SFC conditions: 2% MeOH, 3.0 mL/min, Chiralcel OJ-H column, λ=210 nm, t_{R} (min): major=3.54, minor=3.89.

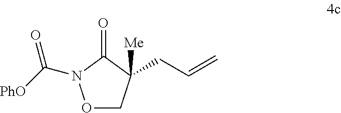 [SMILES: C=CC[C@]1(C)CON(C(=O)Oc2ccccc2)C1=O]

### Isoxazolidinone 4c

Flash column chromatography (SiO₂, 15→20% EtOAc in hexanes) afforded isoxazolidinone 4c (95% yield) as a colorless oil. 73% ee, [α]_{D}²⁵ −28.4 (c 1.14, CHCl₃); R_{f}=0.32 (25% EtOAc in hexanes); ¹H NMR (500 MHz, CDCl₃) δ 7.43-7.38 (m, 2H), 7.30-7.21 (m, 3H), 5.80 (ddt, J=16.6, 10.4, 7.4 Hz, 1H), 5.25-5.19 (m, 2H), 4.38 (d, J=8.7 Hz, 1H), 4.15 (d, J=8.7 Hz, 1H), 2.53-2.40 (m, 2H), 1.36 (s, 3H); ¹³C NMR (126 MHz, CDCl₃) δ 171.7, 150.2, 146.2, 131.6, 129.7, 126.6, 121.4, 120.4, 77.2, 47.3, 39.3, 19.6; IR (Neat Film, NaCl) 1798, 1757, 1494, 1458, 1309, 1274, 1231, 1195, 1162, 1085, 982, 937, 746 cm⁻¹; HRMS (ESI-APCI+) m/z calc'd for C₁₄H₁₆NO₄ [M+H]⁺: 262.1074. found 262.1062; SFC conditions: 5% MeOH, 3.0 mL/min, Chiralpak AD-H column, λ=235 nm, t_{R} (min): major=6.88, minor=8.08.

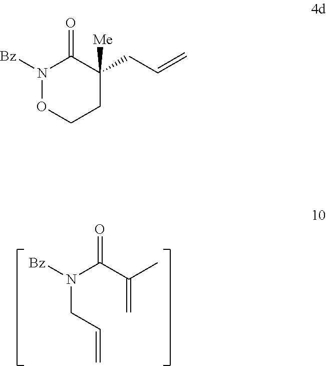

### 1,2-Oxazinan-3-one 4d

Flash column chromatography (SiO₂, 15→20% EtOAc in hexanes) afforded 1,2-oxazinan-3-one 4d (29% yield) as a colorless oil and imide 10 (30% yield) as a colorless oil. 88% ee, [α]_{D}²⁵ −25.8 (c 0.45, CHCl₃); R_{f}=0.38 (20% EtOAc in hexanes); ¹H NMR (500 MHz, CDCl₃) δ 7.64-7.50 (m, 3H), 7.44-7.39 (m, 2H), 5.81 (ddt, J=16.7, 10.3, 7.4 Hz, 1H), 5.20-5.14 (m, 2H), 4.35 (t, J=7.3 Hz, 2H), 2.53 (dt, J=7.4, 1.2 Hz, 2H), 2.23 (dt, J=13.8, 7.4 Hz, 1H), 1.94 (dt, J=13.8, 7.1 Hz, 1H), 1.37 (s, 3H); ¹³C NMR (126 MHz, CDCl₃) δ 175.9, 167.9, 134.4, 133.2, 132.4, 129.0, 128.2, 119.5, 69.9, 43.7, 43.1, 33.3, 24.3; IR (Neat Film, NaCl) 1749, 1700, 1449, 1271, 1207, 1176, 1043, 921 cm⁻¹; HRMS (ESI-APCI+) m/z calc'd for C₁₅H₁₈NO₃ [M+H]⁺: 260.1281. found 260.1275; SFC conditions: 10% IPA, 3.0 mL/min, Chiralcel OD-H column, λ=254 nm, t_{R} (min): major=3.91, minor=3.03.

Imide 10:

¹H NMR (500 MHz, CDCl₃) δ 7.52-7.43 (m, 3H), 7.42-7.35 (m, 2H), 5.97 (ddt, J=17.2, 10.2, 6.2 Hz, 1H), 5.35-5.27 (m, 2H), 5.20 (dq, J=10.2, 1.0 Hz, 1H), 5.13 (m, 1H), 4.52-4.46 (m, 2H), 1.63 (dd, J=1.5, 1.0 Hz, 3H); ¹³C NMR (126 MHz, CDCl₃) δ 174.6, 173.8, 143.4, 137.5, 132.5, 132.0, 128.8, 128.4, 122.2, 118.6, 48.5, 18.5; IR (Neat Film, NaCl) 1698, 1660, 1449, 1337, 1270, 1195, 1099, 930, 801, 706 cm⁻¹; HRMS (ESI-APCI+) m/z calc'd for C₁₄H₁₆NO₂ [M+H]⁺: 230.1176. found 230.1165.

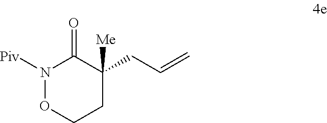

### 1,2-Oxazinan-3-one 4e

Flash column chromatography (SiO₂, 10→15% EtOAc in hexanes) afforded 1,2-oxazinan-3-one 4e (48% yield) as a colorless oil. 73% ee, [α]_{D}²⁵ −26.3 (c 0.50, CHCl₃); R_{f}=0.29 (25% EtOAc in hexanes); ¹H NMR (500 MHz, CDCl₃) δ 5.80 (m, 1H), 5.15-5.08 (m, 2H), 4.23-4.17 (m, 2H), 2.53-2.42 (m, 2H), 2.12 (ddd, J=13.8, 8.1, 6.9 Hz, 1H), 1.86 (ddd, J=13.8, 8.1, 6.8 Hz, 1H), 1.33 (s, 3H), 1.29 (s, 9H); ¹³C NMR (126 MHz, CDCl₃) δ 176.7, 174.8, 133.5, 119.2, 69.3, 43.3, 43.0, 41.6, 32.5, 26.7, 24.3; IR (Neat Film, NaCl) 2975, 2935, 1753, 1708 1462, 1272, 1180, 1131, 917 cm⁻¹; HRMS (ESI-APCI+) m/z calc'd for C₁₃H₂₂NO₃ [M+H]⁺: 240.1594. found 240.1591; SFC conditions: 5% IPA, 2.5 mL/min, Chiralpak AS-H column, λ=210 nm, t_{R} (min): major=7.10, minor=6.65.

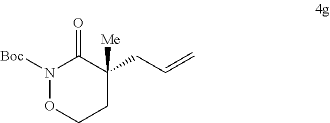 [SMILES: C=CC[C@]1(C)CCON(C(=O)OC(C)(C)C)C1=O]

### 1,2-Oxazinan-3-one 4g

Flash column chromatography (SiO₂, 10→12% EtOAc in hexanes) afforded 1,2-oxazinan-3-one 4g (67% yield) as a colorless oil. 85% ee, [α]_{D}²⁵ −25.4 (c 0.54, CHCl₃); R_{f}=0.47 (33% EtOAc in hexanes); ¹H NMR (500 MHz, CDCl₃) δ 5.80 (ddt, J=16.8, 10.2, 7.4 Hz, 1H), 5.15-5.08 (m, 2H), 4.25-4.19 (m, 2H), 2.49-2.46 (m, 2H), 2.10 (ddd, J=13.8, 8.4, 6.6 Hz, 1H), 1.86 (ddd, J=13.8, 8.5, 6.7 Hz, 1H), 1.55 (s, 9H), 1.31 (s, 3H); ¹³C NMR (126 MHz, CDCl₃) δ 174.3, 148.9, 133.5, 119.1, 84.5, 69.4, 43.7, 42.8, 32.7, 28.2, 24.0; IR (Neat Film, NaCl) 2979, 1744, 1775, 1370, 1281, 1255, 1217, 1156, 1124 cm⁻¹; HRMS (ESI-APCI+) m/z calc'd for C₁₃H₂₂NO₄ [M+H]⁺: 256.1543. found 256.1536; SFC conditions: 1% IPA, 3.0 mL/min, Chiralcel OJ-H column, λ=210 nm, t_{R} (min): major=3.82, minor=3.31.

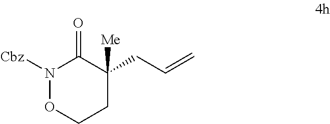 [SMILES: C=CC[C@]1(C)CCON(C(=O)OCc2ccccc2)C1=O]

### 1,2-Oxazinan-3-one 4h

Flash column chromatography (SiO₂, 10→15% EtOAc in hexanes) afforded 1,2-oxazinan-3-one 4h (89% yield) as a colorless oil. 84% ee, [α]_{D}²⁵ −17.4 (c 1.15, CHCl₃); R_{f}=0.24 (20% EtOAc in hexanes); ¹H NMR (500 MHz, CDCl₃) δ 7.46-7.31 (m, 5H), 5.80 (ddt, J=16.6, 10.2, 7.4 Hz, 1H), 5.32 (s, 2H), 5.16-5.09 (m, 2H), 4.29-4.21 (m, 2H), 2.49 (m, 2H), 2.11 (ddd, J=13.8, 8.4, 6.6 Hz, 1H), 1.87 (ddd, J=13.8, 8.5, 6.7 Hz, 1H), 1.33 (s, 3H); ¹³C NMR (126 MHz, CDCl₃) δ 174.1, 150.3, 135.1, 133.3, 128.8, 128.6, 128.4, 119.3, 69.7, 69.0, 43.7, 42.7, 32.5, 24.0; IR (Neat Film, NaCl) 2977, 2939, 1777, 1738, 1456, 1379, 1268, 1217, 1123, 995, 922, 753 cm⁻¹; HRMS (ESI-APCI+) m/z calc'd for C₁₆H₂₀NO₄ [M+H]⁺: 290.1387. found 290.1374; SFC conditions: 5% MeOH, 3.0 mL/min, Chiralpak AD-H column, λ=210 nm, t_{R} (min): major=8.31, minor=7.88.

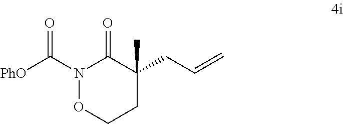 [SMILES: C=CC[C@]1(C)CCON(C(=O)Oc2ccccc2)C1=O]

### 1,2-Oxazinan-3-one 4i

Flash column chromatography (SiO₂, 10→20% EtOAc in hexanes) afforded 1,2-oxazinan-3-one 4i (70% yield) as a colorless oil. 87% ee, [α]_{D}²⁵ −26.2 (c 0.90, CHCl₃); R_{f}=0.31 (25% EtOAc in hexanes); ¹H NMR (500 MHz, CDCl₃) δ 7.42-7.37 (m, 2H), 7.28-7.20 (m, 3H), 5.84 (ddt, J=17.5, 10.3, 7.4 Hz, 1H), 5.20-5.14 (m, 2H), 4.41-4.32 (m, 2H), 2.59-2.50 (m, 2H), 2.20 (ddd, J=14.6, 8.5, 6.5 Hz, 1H), 1.96 (ddd, J=13.9, 8.5, 6.6 Hz, 1H), 1.40 (s, 3H); ¹³C NMR (126 MHz, CDCl₃) δ 174.2, 150.5, 148.7, 133.2, 129.6, 126.4, 121.5, 119.5, 69.8, 43.9, 42.8, 32.5, 24.0; IR (Neat Film, NaCl) 2936, 1786, 1755, 1494, 1269, 1189, 1162, 1102, 934 cm⁻¹; HRMS (ESI-APCI+) m/z calc'd for C₁₅H₁₈NO₄ [M+H]⁺: 276.1230. found 276.1225; SFC conditions: 10% IPA, 2.5 mL/min, Chiralcel OJ-H column, λ=254 nm, t_{R} (min): major=9.61, minor=7.70.

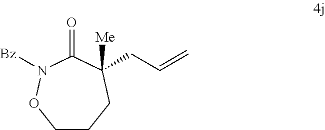

### 1,2-Oxazepan-3-one 4j

Flash column chromatography (SiO₂, 15% EtOAc in hexanes) afforded 1,2-oxazepan-3-one 4j (81% yield) as a colorless oil. 93% ee, [α]_{D}²⁵ −20.6 (c 1.00, CHCl₃); R_{f}=0.56 (33% EtOAc in hexanes); ¹H NMR (500 MHz, CDCl₃) δ 7.57-7.47 (m, 3H), 7.42-7.37 (m, 2H), 5.82 (ddt, J=17.3, 10.2, 7.3 Hz, 1H), 5.20-5.13 (m, 2H), 4.36-4.04 (m, 2H), 2.68 (br s, 1H), 2.47 (dd, J=13.6, 6.7 Hz, 1H), 2.14-1.71 (m, 4H), 1.32 (s, 3H); ¹³C NMR (126 MHz, CDCl₃) δ 178.1, 169.5, 135.1, 133.9, 132.0, 128.3, 128.3, 118.8, 77.4, 47.7, 42.8, 34.3, 25.6, 24.7; IR (Neat Film, NaCl) 2938, 1740, 1699, 1449, 1267, 1210, 1140, 997 cm⁻¹; HRMS (ESI-APCI+) m/z calc'd for C₁₆H₂₀NO₃ [M+H]⁺: 274.1438. found 274.1440; SFC conditions: 5% MeOH, 3.0 mL/min, Chiralcel OJ-H column, λ=254 nm, t_{R} (min): major=5.01, minor=4.30.

Allylic alkylation proceeds with 1,3-oxazinan-4-one 5 as an alternative β-hydroxy acid synthon of 3a. 5 was successfully converted into 6 in 90% yield and 94% ee.

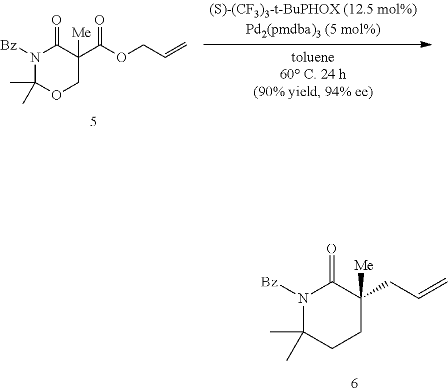

### 1,3-Oxazinan-4-one 6

Flash column chromatography (SiO₂, 10→15% EtOAc in hexanes) afforded 1,3-oxazinan-4-one 6 (90% yield) as a colorless oil. 94% ee, [α]_{D}²⁵ −50.9 (c 1.53, CHCl₃); R_{f}=0.29 (15% EtOAc in hexanes); ¹H NMR (500 MHz, CDCl₃) δ 7.69-7.63 (m, 2H), 7.50 (m, 1H), 7.41 (dd, J=8.4, 7.1 Hz, 2H), 5.77 (ddt, J=16.6, 10.5, 7.3 Hz, 1H), 5.20-5.11 (m, 2H), 3.99 (d, J=12.1 Hz, 1H), 3.74 (d, J=12.1 Hz, 1H), 2.59 (m, 1H), 2.32 (m, 1H), 1.75 (s, 3H), 1.74 (s, 3H), 1.27 (s, 3H); ¹³C NMR (126 MHz, CDCl₃) δ 176.5, 175.7, 136.6, 132.8, 132.5, 128.6, 128.1, 119.5, 92.6, 66.7, 43.1, 40.5, 27.2, 26.9, 21.6; IR (Neat Film, NaCl) 1699, 1683, 1386, 1261, 1174, 1084 cm⁻¹; HRMS (ESI-APCI+) m/z calc'd for C₁₇H₂₂NO₃ [M+H]⁺: 288.1594. found 288.1582; SFC conditions: 2% MeOH, 3.0 mL/min, Chiralcel OJ-H column, λ=254 nm, t_{R} (min): major=4.65, minor=3.14.

### Example 3

### Derivatization of Allylic Alkylation Products

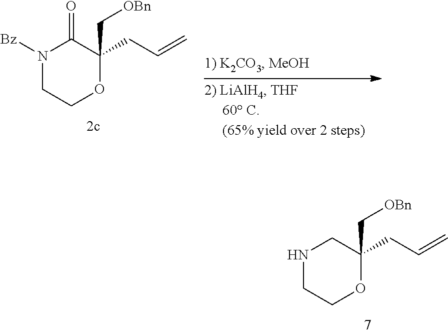

### Morpholine 7

To a solution of 2c (25.3 mg, 69.2 μmol, 1 equiv) in MeOH (0.7 mL) was added K₂CO₃ (1.9 mg, 13.8 μmol, 0.2 equiv) at room temperature. After stirring at room temperature for 2 h, the reaction mixture was filtered and the filtrate was concentrated in vacuo. The residue was used for the next reaction without further purification.

To a solution of the crude morpholinone in THF (2.0 mL) was added LiAlH₄ (7.9 mg, 208 μmol, 3.0 equiv) at room temperature. After stirring at 60° C. for 2 h, H₂O (8 μL), 15% aqueous NaOH (8 μL) and H₂O (24 μL) were added to the reaction mixture sequentially. The resulting mixture was diluted with diethyl ether (30 mL), dried over Na₂SO₄, filtered and the filtrate was concentrated in vacuo. Flash column chromatography (SiO₂, CHCl₃:MeOH:Et₂NH=94:5:1) afforded morpholine 7 (12.4 mg, 45.2 μmol, 65% yield) as a colorless oil. [α]_{D}²⁵ −13.9 (c 0.52, CHCl₃); R_{f}=0.35 (CHCl₃:MeOH:Et₂NH=94:5:1); ¹H NMR (500 MHz, CDCl₃) δ 7.39-7.25 (m, 5H), 5.78 (ddt, J=17.4, 10.2, 7.3 Hz, 1H), 5.14-5.04 (m, 2H), 4.55 (s, 2H), 3.77-3.71 (m, 2H), 3.53 (d, J=9.5 Hz, 1H), 3.47 (d, J=9.5 Hz, 1H), 2.93-2.81 (m, 3H), 2.76 (d, J=12.6 Hz, 1H), 2.59 (m, 1H), 2.35 (m, 1H); ¹³C NMR (126 MHz, CDCl₃) δ 138.4, 133.3, 128.5, 127.8, 127.8, 118.2, 73.7, 73.7, 71.8, 61.8, 50.7, 45.8, 37.6; IR (Neat Film, NaCl) 2933, 2864, 1453, 1101, 1085, 737, 698 cm⁻¹; HRMS (ESI+) m/z calc'd for C₁₅H₂₂NO₂ [M+H]⁺: 248.1645. found 248.1648.

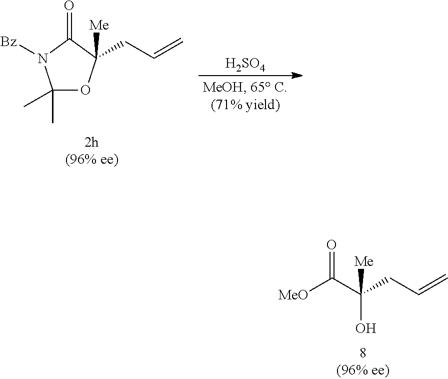

### α-Hydroxyester 8

To a solution of 2h (16.0 mg, 58.5 μmol, 1 equiv) in MeOH (4.0 mL) was added H₂SO₄ (11.5 mg, 117 μmol, 2.0 equiv) at room temperature. After stirring at 65° C. for 48 h, the reaction mixture was quenched with saturated aqueous sodium bicarbonate and diluted with diethyl ether (30 mL). The phases were separated and the aqueous phase was extracted with diethyl ether twice. The combined organic layers were washed with brine, dried over Na₂SO₄, filtered and the filtrate was concentrated in vacuo. Flash column chromatography (SiO₂, 20→50% diethyl ether in hexanes) afforded α-hydroxyester 8 (6.0 mg, 41.6 μmol, 70% yield) as a colorless oil. 96% ee, [α]_{D}²⁵+24.2 (c 0.26, CHCl₃); R_{f}=0.37 (20% EtOAc in hexanes); ¹H NMR (500 MHz, CDCl₃) δ 5.76 (ddt, J=16.8, 10.4, 7.3 Hz, 1H), 5.16-5.07 (m, 2H), 3.77 (s, 3H), 3.10 (s, 1H), 2.50 (m, 1H), 2.39 (m, 1H), 1.42 (s, 3H); ¹³C NMR (126 MHz, CDCl₃) δ 177.1, 132.5, 119.3, 74.7, 52.9, 44.9, 25.6; IR (Neat Film, NaCl) 3504, 2982, 2955, 1736, 1641, 1438, 1272, 1068 cm⁻¹; HRMS (ESI+) m/z calc'd for C₇H₁₃O₃ [M+H]⁺: 145.0865. found 145.0867; chiral GC conditions: 85° C. isotherm, G-TA column, t_{R} (min): major=7.57, minor=7.20.

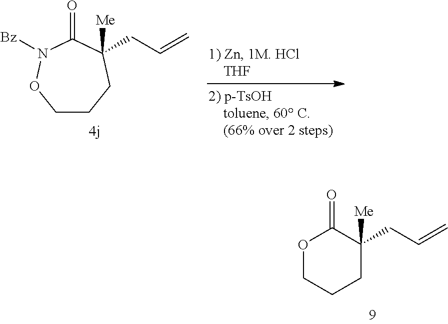

### δ-Lactone 9

To a solution of 4j (15.5 mg, 56.7 μmol, 1 equiv) in THF (1.0 mL) was added Zn (37.1 mg, 567 μmol, 10 equiv) and 1 M HCl (0.1 mL) at room temperature. After stirring at room temperature for 3 h, the reaction mixture was quenched with 1 M HCl and diluted with ethyl acetate (30 mL). The phases were separated and the aqueous phase was extracted with ethyl acetate twice. The combined organic layers were washed with brine, dried over Na₂SO₄, filtered and the filtrate was concentrated in vacuo. The residue was used for the next reaction without further purification.

To a solution of the crude alcohol in toluene (2 mL) was added p-toluenesulfonic acid monohydrate (12.9 mg, 68.0 μmol, 1.2 equiv) at room temperature. After stirring at 60° C. for 30 min, the reaction mixture was diluted with diethyl ether (30 mL). The organic phase was washed with saturated aqueous sodium bicarbonate and brine, dried over Na₂SO₄, filtered, and the filtrate was concentrated in vacuo. Flash column chromatography (SiO₂, 20→30% diethyl ether in hexanes) afforded 6-lactone 9 (5.8 mg, 37.6 μmol, 66% yield) as a colorless oil. [α]_{D}²⁵ −29.8 (c 0.28, CHCl₃); R_{f}=0.36 (25% EtOAc in hexanes); ¹H NMR (500 MHz, CDCl₃) δ 5.75 (dddd, J=16.9, 10.2, 8.0, 6.7 Hz, 1H), 5.17-5.07 (m, 2H), 4.36 (m, 1H), 4.27 (m, 1H), 2.56 (m, 1H), 2.22 (m, 1H), 1.98-1.77 (m, 3H), 1.63 (m, 1H), 1.29 (s, 3H); ¹³C NMR (126 MHz, CDCl₃) δ 176.3, 133.4, 119.3, 70.6, 44.6, 42.4, 31.8, 26.5, 20.7; IR (Neat Film, NaCl) 2936, 1725, 1131 cm⁻¹; HRMS (ESI+) m/z calc'd for C₉H₁₅NO₂ [M+H]⁺: 155.1067. found 155.1068.

### Example 4

### Methods for Determining Enantiomeric Excess of Alkylation Products

| entry | compound | | analytic conditions | ee (%) | polarimetry |
|---|---|---|---|---|---|
| 1 | 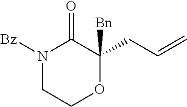 | 2b | SFC: 10% MeOH, 3.0 mL/min Chiralpak AD-H, λ = 254 nm t_{R} (min): major 3.67, minor 5.93 | 99 | [α]_{D}²⁵ +85.9 (c 1.15, CHCl₃) |
| 2 | 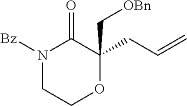 | 2c | SFC: 5% MeOH, 3.0 mL/min Chiralpak AD-H, λ = 254 nm t_{R} (min): major 5.91, minor 6.73 | 99 | [α]_{D}²⁵ −22.4 (c 1.37, CHCl₃) |
| 3 | 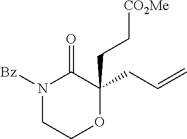 | 2d | SFC: 5% MeOH, 3.0 mL/min Chiralcel OD-H, λ = 254 nm t_{R} (min): major 6.16, minor 5.66 | 99 | [α]_{D}²⁵ +24.6 (c 1.15, CHCl₃) |
| 4 | 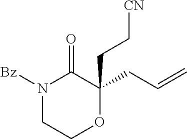 | 2e | SFC: 3% MeOH, 2.5 mL/min Chiralpak AS-H, λ = 254 nm t_{R} (min): major 5.79, minor 6.53 | 99 | [α]_{D}²⁵ +38.5 (c 1.24, CHCl₃) |
| 5 | 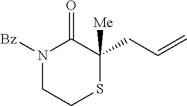 | 2f | SFC: 5% MeOH, 3.0 mL/min Chiralcel OJ-H, λ = 254 nm t_{R} (min): major 6.80, minor 5.74 | 86 | [α]_{D}²⁵ −45.8 (c 1.35, CHCl₃) |
| 6 | 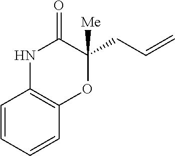 | SI-24 | SFC: 5% IPA, 3.0 mL/min Chiralcel OJ-H, λ = 210 nm t_{R} (min): major 4.18, minor 4.64 | 95 | [α]_{D}²⁵ −15.2 (c 0.21, CHCl₃) |
| 7 | 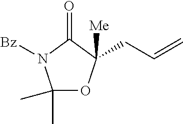 | 2h | SFC: 2% IPA, 3.0 mL/min Chiralcel OJ-H, λ = 254 nm t_{R} (min): major 5.59, minor 3.96 | 96 | [α]_{D}²⁵ +68.2 (c 1.05, CHCl₃) |
| 8 | 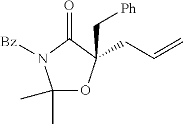 | 2i | SFC: 5% MeOH, 3.0 mL/min Chiralcel OJ-H, λ = 254 nm t_{R} (min): major 3.51, minor 4.44 | 92 | [α]_{D}²⁵ +46.9 (c 1.03, CHCl₃) |
| 9 | 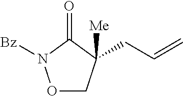 | 4a | SFC: 5% MeOH, 3.0 mL/min Chiralcel OJ-H, λ = 254 nm t_{R} (min): major 8.07, minor 5.70 | 73 | [α]_{D}²⁵ −33.5 (c 1.05, CHCl₃) |
| 10 | 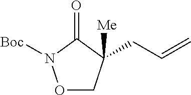 [SMILES: C=CC[C@]1(C)CON(C(=O)OC(C)(C)C)C1=O] | 4b | SFC: 2% MeOH, 3.0 mL/min Chiralcel OJ-H, λ = 210 nm t_{R} (min): major 3.54, minor 3.89 | 72 | [α]_{D}²⁵ −27.7 (c 1.10, CHCl₃) |
| 11 | 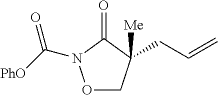 [SMILES: C=CC[C@]1(C)CON(C(=O)Oc2ccccc2)C1=O] | 4c | SFC: 5% MeOH, 3.0 mL/min Chiralpak AD-H, λ = 235 nm t_{R} (min): major 6.88, minor 8.08 | 73 | [α]_{D}²⁵ −28.4 (c 1.14, CHCl₃) |
| 12 | 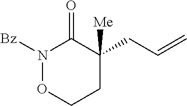 | 4d | SFC: 10% IPA, 3.0 mL/min Chiralcel OD-H, λ = 254 nm t_{R} (min): major 3.91, minor 3.03 | 88 | [α]_{D}²⁵ −28.4 (c 0.45, CHCl₃) |
| 13 | 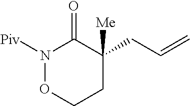 | 4e | SFC: 5% IPA, 2.5 mL/min Chiralpak AS-H, λ = 210 nm t_{R} (min): major 7.10, minor 6.65 | 73 | [α]_{D}²⁵ −26.3 (c 0.50, CHCl₃) |
| 14 | 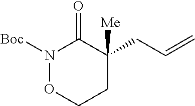 [SMILES: C=CC[C@]1(C)CCON(C(=O)OC(C)(C)C)C1=O] | 4g | SFC: 1% IPA, 3.0 mL/min Chiralcel OJ-H, λ = 210 nm t_{R} (min): major 3.82, minor 3.31 | 85 | [α]_{D}²⁵ −25.4 (c 0.54, CHCl₃) |
| 15 | 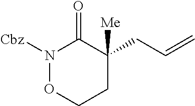 [SMILES: C=CC[C@]1(C)CCON(C(=O)OCc2ccccc2)C1=O] | 4h | SFC: 5% MeOH, 3.0 mL/min Chiralpak AD-H, λ = 210 nm t_{R} (min): major 8.31, minor 7.88 | 84 | [α]_{D}²⁵ −17.4 (c 1.15, CHCl₃) |
| 16 | 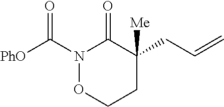 [SMILES: C=CC[C@]1(C)CCON(C(=O)Oc2ccccc2)C1=O] | 4i | SFC: 10% IPA, 2.5 mL/min Chiralcel OJ-H, λ = 254 nm t_{R} (min): major 9.61, minor 7.70 | 87 | [α]_{D}²⁵ −26.2 (c 0.90, CHCl₃) |
| 17 | 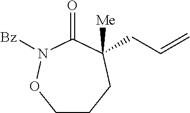 | 4j | SFC: 5% MeOH, 3.0 mL/min Chiralcel OJ-H, λ = 254 nm t_{R} (min): major 5.01, minor 4.30 | 93 | [α]_{D}²⁵ −20.6 (c 1.00, CHCl₃) |
| 18 | 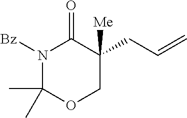 | 6 | SFC: 2% MeOH, 3.0 mL/min Chiralcel OJ-H, λ = 254 nm t_{R} (min): major 4.65, minor 3.14 | 94 | [α]_{D}²⁵ −50.9 (c 1.53, CHCl₃) |
| 19 | 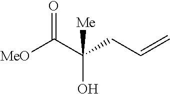 | 8 | GC: 85° C. isotherm G-TA column t_{R} (min): major 7.57, minor 7.20 | 96 | [α]_{D}²⁵ +24.2 (c 0.26, CHCl₃) |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound represented by formula (I), 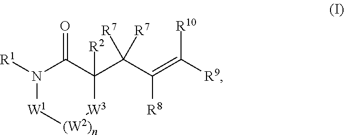
R¹ represents hydrogen or optionally substituted alkyl, cycloalkyl, (cycloalkyl)alkyl, aryl, aralkyl, heteroaryl, heteroaralkyl, alkenyl, alkynyl, —C(O)alkyl, —C(O)aryl, —C(O)aralkyl, —C(O)heteroaryl, —C(O)heteroaralkyl, —C(O)O(alkyl), —C(O)O(aryl), C(O)O(aralkyl), —C(O)O(heteroaryl), or —C(O)O(heteroaralkyl); or
R² represents hydrogen or substituted or unsubstituted alkyl, aralkyl, aryl, heteroaralkyl, heteroaryl, alkenyl, alkynyl, or halo;
R⁷, R⁸, R⁹, and R¹⁰ are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
W¹, W², and W³ are independently selected for each occurrence from CR¹¹R¹², O, and S;
R¹¹ and R¹² are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
wherein any occurrence of may optionally combine with any second occurrence of or an occurrence of R¹², along with the carbons to which they are attached, to form an optionally substituted 3-8 membered ring; and
n is an integer from 0-3;
wherein the compound of formula (I) is not

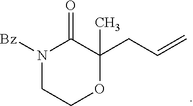

2.-25. (canceled)

26. A compound represented by formula (II), 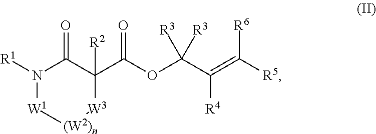
or a tautomer and/or salt thereof, wherein:
R¹ represents hydrogen or optionally substituted alkyl, cycloalkyl, (cycloalkyl)alkyl, aryl, aralkyl, heteroaryl, heteroaralkyl, alkenyl, alkynyl, —C(O)alkyl, —C(O)aryl, —C(O)aralkyl, —C(O)heteroaryl, —C(O)heteroaralkyl, —C(O)O(alkyl), —C(O)O(aryl), C(O)O(aralkyl), —C(O)O(heteroaryl), or —C(O)O(heteroaralkyl); or
R² represents hydrogen or substituted or unsubstituted alkyl, aralkyl, aryl, heteroaralkyl, heteroaryl, alkenyl, alkynyl, or halo;
R³, R⁴, R⁵, and R⁶ are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
W², and W³ are independently selected for each occurrence from CR¹¹R¹², O, and S;
R¹¹ and R¹² are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
wherein any occurrence of may optionally combine with any second occurrence of or an occurrence of R¹², along with the carbons to which they are attached, to form an optionally substituted 3-8 membered ring; and
n is an integer from 0-3;
wherein the compound is not

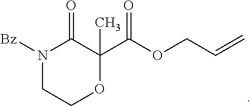

27. A method for the preparation of a compound of formula (I): 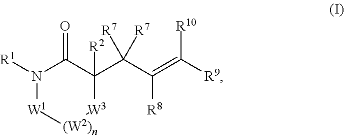
comprising treating a compound of formula (II): 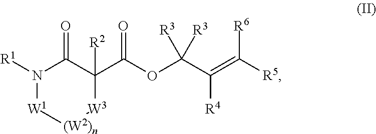
with a transition metal catalyst under alkylation conditions, wherein, as valence and stability permit,
R¹ represents hydrogen or optionally substituted alkyl, cycloalkyl, (cycloalkyl)alkyl, aryl, aralkyl, heteroaryl, heteroaralkyl, alkenyl, alkynyl, —C(O)alkyl, —C(O)aryl, —C(O)aralkyl, —C(O)heteroaryl, —C(O)heteroaralkyl, —C(O)O(alkyl), —C(O)O(aryl), C(O)O(aralkyl), —C(O)O(heteroaryl), or —C(O)O(heteroaralkyl); or
R² represents hydrogen or substituted or unsubstituted alkyl, aralkyl, aryl, heteroaralkyl, heteroaryl, alkenyl, alkynyl, or halo;
R³, R⁴, R⁵, R⁶, R⁷, R⁸, R⁹, and R¹⁰ are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
W¹, W², and W³ are independently selected for each occurrence from CR¹¹R¹², O, and S;
R¹¹ and R¹² are independently selected for each occurrence from hydrogen, hydroxyl, halogen, nitro, alkyl, alkenyl, alkynyl, cyano, carboxyl, sulfate, amino, alkoxy, alkylamino, alkylthio, hydroxyalkyl, alkoxyalkyl, aminoalkyl, thioalkyl, ether, thioether, ester, amide, thioester, carbonate, carbamate, urea, sulfonate, sulfone, sulfoxide, sulfonamide, acyl, acyloxy, acylamino, aryl, heteroaryl, carbocyclyl, heterocyclyl, aralkyl, arylalkoxy, heteroaralkyl, carbocyclylalkyl, and heterocyclylalkyl;
wherein any occurrence of may optionally combine with any second occurrence of or an occurrence of R¹², along with the carbons to which they are attached, to form an optionally substituted 3-8 membered ring; and
n is an integer from 0-3;
wherein the compound of formula (I) is not

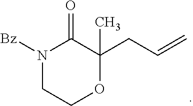

28. The method of claim 27, wherein W¹ is O.

29. The method of claim 28, wherein W² and W³ each represent CR¹¹R¹².

30. The method of claim 29, wherein W² and W³ each represent CH₂.

31. The method of claim 28, wherein n is 0, 1, or 2.

32. (canceled)

33. (canceled)

34. The method of claim 27, wherein W² is O and n is 1.

35. The method of claim 34, wherein W¹ and W³ each represent CR¹¹R¹².

36. The method of claim 35, wherein at least one occurrence of or R¹² is not hydrogen.

37. The method of claim 27, wherein W³ is O.

38. The method of claim 37, wherein W¹ and W² each represent CR¹¹R¹².

39. The method of claim 38, wherein at least one occurrence of or R¹² is not hydrogen.

40. The method of claim 37, wherein n is 0, 1, or 2.

41.-43. (canceled)

44. The method of claim 27, wherein R³, R⁴, R⁵, R⁶, R⁷, R⁸, R⁹, and R¹⁰ are each hydrogen.

45. (canceled)

46. The method of claim 27, wherein R² is alkyl, optionally substituted with halo, hydroxy, alkoxy, aryloxy, arylalkoxy, cyano, nitro, azido, —CO₂H, —C(O)O(alkyl), or amino.

47. (canceled)

48. The method of claim 27, wherein R¹ represents optionally substituted —C(O)alkyl, —C(O)aryl, —C(O)aralkyl, —C(O)heteroaryl, —C(O)heteroaralkyl, —C(O)O(alkyl), —C(O)O(aryl), C(O)O(aralkyl), —C(O)O(heteroaryl), or —C(O)O(heteroaralkyl).

49. The method of claim 27, wherein R¹¹ and R¹² are each selected, independently for each occurrence, from hydrogen, halogen, cyano, alkyl, alkoxy, alkylthio, amide, amine, aryloxy, and arylalkoxy.

50. The method of claim 49, wherein R¹¹ and R¹² are each selected, independently for each occurrence, from hydrogen, alkyl, alkoxy, and alkylthio.

51. The method of claim 27, wherein the transition metal catalyst comprises a transition metal selected from palladium, nickel, and platinum.

52.-55. (canceled)

56. The method of claim 27, wherein the transition metal catalyst further comprises a chiral ligand.

57. The method of claim 56, wherein the chiral ligand is an enantioenriched phosphine ligand.

58. The method of claim 57, wherein the enantioenriched phosphine ligand is a phosphinooxazoline ligand.

59.-61. (canceled)

62. The method of claim 27, whereby the compound of formula (I) is enantioenriched.
